SEQUENCE LISTING (40 sequences)

SEQ ID NO: 1 - protein, 592 aa, Geobacillus stearothermophilus
  Geobacillus strain
MESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 2 - DNA, 26 bp, Artificial Sequence
  E_Sarbeco_F1
acaggtacgttaatagttaatagcgt

SEQ ID NO: 3 - DNA, 22 bp, Artificial Sequence
  E_Sarbeco_R2
atattgcagcagtacgcacaca

SEQ ID NO: 4 - DNA, 26 bp, Artificial Sequence
  E_Sarbeco_P1
acactagccatccttactgcgcttcg

SEQ ID NO: 5 - DNA, 16 bp, Artificial Sequence
  MS2_F
tgctcgcggatacccg

SEQ ID NO: 6 - DNA, 20 bp, Artificial Sequence
  MS2_R
aacttgcgttctcgagcgat

SEQ ID NO: 7 - DNA, 24 bp, Artificial Sequence
  MS2_P
acctcgggtttccgtcttgctcgt

SEQ ID NO: 8 - DNA, 22 bp, Artificial Sequence
  RT Primer
gtaagaacacctgtgcctgtta

SEQ ID NO: 9 - DNA, 55 bp, Artificial Sequence
  Forward Primer
acactctttccctacacgacgctcttccgatctagactttttaggtccacaaaca

SEQ ID NO: 10 - DNA, 56 bp, Artificial Sequence
  Reverse Primer
tgactggagttcagacgtgtgctcttccgatctccggtagcacaccttgtaatggt

SEQ ID NO: 11 - DNA, 51 bp, Artificial Sequence
  SARS-CoV-2-FIP-BC
tgcggccaatgtttgtaatcagagcttacactccaaggaaattttggggac

SEQ ID NO: 12 - DNA, 39 bp, Artificial Sequence
  SARS-CoV-2-BIP
cgcattggcatggaagtcactttgatggcacctgtgtag

SEQ ID NO: 13 - DNA, 19 bp, Artificial Sequence
  SARS-CoV-2-LF
ttccttgtctgattagttc

SEQ ID NO: 14 - DNA, 18 bp, Artificial Sequence
  SARS-CoV-2-LB
accttcgggaacgtggtt

SEQ ID NO: 15 - DNA, 18 bp, Artificial Sequence
  SARS-CoV-2-F3
aacacaagctttcggcag

SEQ ID NO: 16 - DNA, 22 bp, Artificial Sequence
  SARS-CoV-2-B3
gaaatttggatctttgtcatcc

SEQ ID NO: 17 - DNA, 18 bp, Artificial Sequence
  SARS-CoV-2-F3-LNA
aacacaagctttcggcag

SEQ ID NO: 18 - DNA, 22 bp, Artificial Sequence
  SARS-CoV-2-B3-LNA
gaaatttggatctttgtcatcc

SEQ ID NO: 19 - DNA, 56 bp, Artificial Sequence
  SARS-CoV-2-PCR forward
acactctttccctacacgacgctcttccgatctaacgctgaagcgctgggggcaaa

SEQ ID NO: 20 - DNA, 56 bp, Artificial Sequence
  SARS-CoV-2-PCR reverse
tgactggagttcagacgtgtgctcttccgatctgtttgtaatcagttccttgtctg

SEQ ID NO: 21 - DNA, 49 bp, Artificial Sequence
  Influenza-A-FIP
  variation 23: /replace=“a,c,g,t”
  variation 24: /replace=“a,c,g,t”
  variation 25: /replace=“a,c,g,t”
  variation 26: /replace=“a,c,g,t”
  variation 27: /replace=“a,c,g,t”
  variation 28: /replace=“a,c,g,t”
  variation 29: /replace=“a,c,g,t”
  variation 30: /replace=“a,c,g,t”
  variation 31: /replace=“a,c,g,t”
  variation 32: /replace=“a,c,g,t”
ttagtcagaggtgacarrattgnnnnnnnnnncagatcttgaggctctc

SEQ ID NO: 22 - DNA, 39 bp, Artificial Sequence
  Influenza-A-BIP
ttgtkttcacgctcaccgtgtttggacaaagcgtctacg

SEQ ID NO: 23 - DNA, 16 bp, Artificial Sequence
  Influenza-A-LF
gtcttgtctttagcca

SEQ ID NO: 24 - DNA, 17 bp, Artificial Sequence
  Influenza-A-LB
cmagtgagcgaggactg

SEQ ID NO: 25 - DNA, 19 bp, Artificial Sequence
  Influenza-A-F3-1 5x LNA
gacttgaagatgtctttgc

SEQ ID NO: 26 - DNA, 19 bp, Artificial Sequence
  Influenza-A-F3-2 5x LNA
gactggaaagtgtctttgc

SEQ ID NO: 27 - DNA, 19 bp, Artificial Sequence
  Influenza-A-B3-1 5x LNA
trttatttgggtctccatt

SEQ ID NO: 28 - DNA, 19 bp, Artificial Sequence
  Influenza-A-B3-2 5x LNA
trttgtttgggtccccatt

SEQ ID NO: 29 - DNA, 159 bp, SARS-CoV-2
  SARS-CoV-2 spike gene
agactttttaggtccacaaacagttgctggtgcatgtagaagttcaaaagaaagtactactactctgttatggtttggtaaccaacaccataagtgtggtttggaaaccatatgtattgttaaaggaaagtaacaattaaaacccttcaaccacccatt

SEQ ID NO: 30 - protein, 611 aa, Geobacillus stearothermophilus
  Geobacillus strain
MKHHHHHHSAGLEVLFQGPMESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 31 - DNA, 51 bp, Artificial Sequence
  Alternative SARS-CoV-2-FIP-BC
  variation 23: /replace=“a,c,g,t”
  variation 24: /replace=“a,c,g,t”
  variation 25: /replace=“a,c,g,t”
  variation 26: /replace=“a,c,g,t”
  variation 27: /replace=“a,c,g,t”
  variation 28: /replace=“a,c,g,t”
  variation 29: /replace=“a,c,g,t”
  variation 30: /replace=“a,c,g,t”
  variation 31: /replace=“a,c,g,t”
  variation 32: /replace=“a,c,g,t”
gctgcctggagttgaatttcttnnnnnnnnnncaagcctcttctcgttcct

SEQ ID NO: 32 - DNA, 39 bp, Artificial Sequence
  Alternative SARS-CoV-2-BIP
ctgctcttgctttgctgctgtaccagacattttgctctc

SEQ ID NO: 33 - DNA, 19 bp, Artificial Sequence
  Alternative SARS-CoV-2-LF
gaactgttgcgactacgtg

SEQ ID NO: 34 - DNA, 18 bp, Artificial Sequence
  Alternative SARS-CoV-2-LB
cttgacagattgaaccag

SEQ ID NO: 35 - DNA, 18 bp, Artificial Sequence
  Alternative SARS-CoV-2-F3 LNA
aaaggcttctacgcagaa

SEQ ID NO: 36 - DNA, 22 bp, Artificial Sequence
  Alternative SARS-CoV-2-B3 LNA
tttggccttgttgttgttggcc

SEQ ID NO: 37 - DNA, 18 bp, Artificial Sequence
  Alternative SARS-CoV-2-F3 no LNA
aaaggcttctacgcagaa

SEQ ID NO: 38 - DNA, 22 bp, Artificial Sequence
  Alternative SARS-CoV-2-B3 no LNA
tttggccttgttgttgttggcc

SEQ ID NO: 39 - DNA, 55 bp, Artificial Sequence
  Alternative SARS-CoV-2-PCR fwd
acactctttccctacacgacgctcttccgatctcattgccagccattctagcagg

SEQ ID NO: 40 - DNA, 56 bp, Artificial Sequence
  Alternative SARS-CoV-2-PCR rev
tgactggagttcagacgtgtgctcttccgatctgttgaatttcttgaactgttgcg

(19) United States
(12) Patent Application Publication
(10) Pub. No.: US 2024/0271197 A1
(43) Pub. Date: Aug. 15, 2024

(54) RT-LAMP SENSITIVITY INCREASE USING UNPURIFIED BIOSAMPLES

(71) Applicant: UNIVERSITÄTSKLINIKUM BONN (UKB), Bonn (DE)

(72) Inventors: Jonathan SCHMID-BURGK, Bonn (DE); Kerstin LUDWIG, Bonn (DE)

(21) Appl. No.: 18/568,710

(22) PCT Filed: Jun. 9, 2022
(86) PCT No.: PCT/EP2022/065751

§ 371 Date: Dec. 8, 2023

(30) Foreign Application Priority Data
Jun. 10, 2021 (EP) 21178783.3

(51) Int. Cl. C12Q 1/6844; C12Q 1/70

(52) U.S. Cl. CPC C12Q 1/6844; C12Q 1/701

(57) ABSTRACT

The present invention relates to a method for the detection of a target nucleic acid sequence in a sample, wherein the method comprises: a) optionally contacting the sample with a lysis buffer under conditions wherein the sample is lysed, b) optionally heating the lysis buffer; c) subjecting the lysate as obtained after step a) and optionally after step b) or the sample to an isothermal amplification reaction at a temperature of 30 to 75° C., preferably 45 to 75°, more preferably 60 to 70° C., even more preferred about 65° C. with at least two primers specifically amplifying the target nucleic acid sequence, wherein the nucleotides of at least one primer comprise at least two locked nucleic acids (LNAs) which are not directly adjacent to each other within the nucleotides of the at least one primer, and wherein the isothermal amplification reaction comprises a DNA polymerase with reverse transcriptase activity and strand displacement activity and does not comprise a thermostable reverse transcriptase; and d) detecting the presence of the target nucleic acid sequence in the amplification product obtained after or during step d).

The present invention relates to a method for the detection of a target nucleic acid sequence in a sample, wherein the method comprises: a) optionally contacting the sample with a lysis buffer under conditions wherein the sample is lysed, b) optionally heating the lysis buffer; c) subjecting the lysate as obtained after step a) and optionally after step b) or the sample to an isothermal amplification reaction at a temperature of 30 to 75° C., preferably 45 to 75°, more preferably 60 to 70° C., even more preferred about 65° C. with at least two primers specifically amplifying the target nucleic acid sequence, wherein the nucleotides of at least one primer comprise at least two locked nucleic acids (LNAs) which are not directly adjacent to each other within the nucleotides of the at least one primer, and wherein the isothermal amplification reaction comprises a DNA polymerase with reverse transcriptase activity and strand displacement activity and does not comprise a thermostable reverse transcriptase; and d) detecting the presence of the target nucleic acid sequence in the amplification product obtained after or during step d).

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

As of June 2021, the global spread of a novel coronavirus, severe acute respiratory syndrome coronavirus 2 (SARS-CoV-2), has resulted in over about 175 million confirmed cases and about 3.8 million deaths attributed to COVID-19 [Johns Hopkins Center for Systems Science and Engineering (CSSE) COVID tracker] (1). While the SARS-CoV-2 pandemic could be slowed by restrictive isolation measures (2), these place an enormous burden on societies and economies, and, once lifted, exponential spread resumes (1, 3).

Current containment strategies based on “test-trace-isolate” face major issues: (i) many infected individuals do not show any symptoms and therefore remain untested (4); (ii) a shortage of reagents, consumables and instrumentation (e.g., RT-PCR machines) still limits testing capacity with an increasing testing backlog, which prevents broad, timely diagnosis; and (iii) the successive (rather than parallel) testing of close contacts causes a substantial lag in identifying infection chains, resulting in undetected spread due to delayed diagnosis. By contrast, repeated population-scale testing that enables identification of all infected individuals regardless of their symptomatic or contact status is predicted to be an effective measure to help combat the transmission of SARS-CoV-2 (5, 6), pinpoint outbreak areas, and enable local epidemiological interventions that maximize human health, while minimizing the societal impact of restrictive isolation measures.

The current gold-standard test for detection of active SARS-CoV-2 infection is viral RNA extraction from a bio-specimen followed by RT-qPCR to amplify and detect several highly conserved regions of the SARS-CoV-2 genome. However, the global capacity for testing using these approaches is limited in several ways. First, access to reagents, consumables and instruments is limited due to the surge in demand, especially plasticware and RT-PCR instrumentation. Second, most protocols include several hands-on steps that must be performed by trained professionals, which hampers their scalability even when automated systems are used. Third, while several sequencing-based approaches have been proposed (7-9), these are constrained by the need for individual RNA extraction or thermocycling, or their limited sensitivity in detecting low viral loads in clinical specimen. Collectively, these limitations prevent massive, repeated population testing.

Hence, the current COVID 19 pandemic has shown that there is an urgent need for diagnostic methods that enable sensitive, scalable, and multiplexed population-scale testing for the occurrence of a disease in a population. This need is addressed by the present invention.

The present invention therefore relates in a first aspect to a method for the detection of a target nucleic acid sequence in a sample, wherein the method comprises: a) optionally contacting the sample with a lysis buffer under conditions wherein the sample is lysed, b) optionally heating the lysis buffer; c) subjecting the lysate as obtained after step a) (if step a) has been carried out) and optionally after step b) or the sample (if step a) and the optional step have not been carried out)) to an isothermal amplification reaction at a temperature of 30 to 75° C., preferably 45 to 75°, more preferably 60 to 70° C., even more preferred about 65° C. with at least two primers specifically amplifying the target nucleic acid sequence, wherein the nucleotides of at least one primer comprise at least two locked nucleic acids (LNAs) which are not directly adjacent to each other within the nucleotides of the at least one primer, and wherein the isothermal amplification reaction comprises a DNA polymerase with reverse transcriptase activity and strand displacement activity and does not comprise a thermostable reverse transcriptase; and d) detecting the presence of the target nucleic acid sequence in the amplification product obtained after or during step d).

The target nucleic acid sequence can be any nucleic acid sequence the presence or absence of which in a sample is of interest. The target nucleic acid sequence is preferably a target nucleic acid sequence the presence of which in a sample is indicative for the occurrence of a disease or disease state in the subject from which the sample has been obtained. The disease is preferably an infectious disease, such as a bacterial and viral infection, and is preferably a viral infection. The subject is preferably a mammal and most preferably human.

The target nucleic acid sequence is preferably in accordance with the invention is DNA or RNA and is preferably RNA. In the method of the invention the target RNA is reversely transcribed into DNA by the reverse transcriptase activity of the DNA polymerase. The reversely transcribed DNA (also called copy DNA or cDNA) is then amplified in step (c). Regarding DNA it noted that the LNA modified primers as described herein might also help to detect DNA, as they might enable invading double stranded DNA, and might aid in the first steps of LAMP.

Also the sample can be any sample, such as a naturally occurring sample (e.g. food, beverage, a soil sample, a plant sample or sample obtained from a subject) or an industrial sample (e.g. a sample from the production of food, beverage, a medicament or a cosmetic). The sample is preferably a sample that has been obtained from a subject and/or the sample preferably comprises cells.

The optional lysis means breaking down the membrane of a cell. A lysis buffer is accordingly a buffer being capable of breaking down the membrane of a cell after it has been contacted with a sample comprising cells. Most lysis buffers contain buffering salts (e.g. Tris-HCl) and ionic salts (e.g. NaCl) to regulate the pH and osmolarity of the lysate. Sometimes detergents (such as Triton X-100 or SDS) are added to break up membrane structures. Sometimes also enzymes (such as proteinase K) are added. Proteinase K is known to digest RNases and, thus, may be helpful in order to maintain the integrity of the target nucleic acid in the sample. It is believed that the step of contacting the lysis buffer is not only needed to break-up the cells in case the sample comprises cells but is generally helpful in order to make the target nucleic acid sequence more amenable for and/or more stable in the isothermal amplification reaction as defined in step c) of the methods described herein. In more detail, the lysis buffer may make a target nucleic acid accessible by breaking up a virus or other pathogen or a cell and/or make target nucleic acid more stable, for instance, by inhibiting components, such as enzymes in the sample. Preferred compositions of a lysis buffer will be discussed herein below. The lysis step is preferably included in the method of the invention.

The optional heat treatment may be used to inactive the activity of enzymes (DNases and/or RNases) in the sample and/or the lysis buffer (Proteinase K), if present. Incubation at 65° C. for 20 minutes inactivates the majority of enzymes that have an optimal incubation temperature of 37° C. (human body temperature). Enzymes that cannot be inactivated at 65° C. can often be inactivated by incubation at 80° C. for 20 minutes. Thermolabile Proteinase K can, for example, be inactivated by incubation at 55° C. for 10 minutes. Proteinase K can also be inactivated by a Proteinase K inhibitor instead of by heat.

In case no lysis step (and the optional heat treatment) is included in the method of the invention, the method starts with the isothermal amplification reaction of step c) and the sample is in this case directly subjected to the isothermal amplification reaction. This scenario is particularly suitable if the sample is an RNA sample, such as purified RNA. For such sample no lysis step is required. The purified RNA may be stored (e.g. at −80° C.) until its use in the method of the invention.

Isothermal amplification reactions are reactions that amplify a target nucleic acid at a single-temperature. Isothermal amplification methods provide detection of a nucleic acid target sequence in a streamlined, exponential manner, and are not limited by the constraint of thermal cycling, such as classical Taq-PCR. All isothermal amplification reactions have important features in common. For example, because the DNA strands are not heat denatured, all isothermal methods rely on an alternative approach to enable primer binding and initiation of the amplification reaction: a polymerase with strand-displacement activity. Once the reaction is initiated, the polymerase must also separate the strand that is still annealed to the sequence of interest. Isothermal amplification chemistry has been applied to diagnostics with great success and is utilized in several commercial molecular diagnostic platforms, serving large testing centers and point-of-care markets.

Also, real-time readings of amplification are possible with isothermal amplification reactions. Amplified products can be detected, for example, by measuring turbidity or by visual inspection for colour change. This capability eliminates the need for gel electrophoresis. Isothermal amplification reactions could become more popular than PCR soon, owing to their low energy requirement and simplicity which allows for their possible integration into simple, compact systems.

Non-limiting but preferred examples of isothermal amplification reactions are Loop-Mediated Isothermal Amplification (LAMP), Whole Genome Amplification (WGA), Rolling Circle Amplification (RCA), Strand Displacement Amplification (SDA), Helicase-Dependent Amplification (HDA), Recombinase Polymerase Amplification (RPA), Sequence Mediated Amplification of RNA Technology (SMART), Multiple Cross Displacement Amplification (MCDA) and Nucleic Acid Sequences Based Amplification (NASBA); see Zhao et al. (2015), Chem. Rev. 2015, 115, 22, 12491-12545 and Obande and Singh (2020), Infection and Drug Resistance, 13:155-483.

The single-temperature at which the isothermal amplification reaction is carried out is in accordance with the method of the invention 30 to 75° C., preferably 45 to 75°, preferably 60 to 70° C., and even more preferred about 65° C. Unless defined otherwise herein in a particular context, the term “about” as used herein preferably means ±5%, more preferably ±3%, and most preferably ±1%.

While the different isothermal amplification reaction methods differ from one another by the number of primers all these reactions rely on the use of primers for the amplification of the target nucleic acid sequence. A primer generally designates a short synthetic single-stranded nucleic acid sequence being utilized to direct DNA elongation of the target sequence being amplified.

At least one of the primers to be used in accordance with the method of the invention in an isothermal amplification reaction comprises at least two locked nucleic acids (LNAs) which are not directly adjacent to each other within the nucleotides of the at least one primer. The term “which are not directly adjacent to each other within the nucleotides of the at least one primer” means that two consecutive nucleotides in the primer cannot be LNAs. After and in front of each LNA—unless the LNA is the most 5′ or 3′ nucleotide—a non-LNA nucleotide, preferably a deoxynucleotide selected from A, C, T and G is present within the primer. Preferably every second nucleotide along the primer is an LNA. The at least one primer preferably means at least two different primers and most preferably two different primers. The at least two locked nucleic acids LNAs are with increasing preference at least three, at least four and at least five LNAs or are preferably three to six LNAs. The number of LNAs per primer is most preferably five. Preferably the second, fourth, and optionally also one or more of the sixth, eight, tenth and twelfth nucleotide position in the primer from the 5′-end are LNAs. It is in addition preferred with increasing preference that at the 3′-end of the primer the at least two, at least three, at least four or at least five most 3′-terminal nucleotides cannot be LNAs.

A locked nucleic acids (LNAs) (also known as bridged nucleic acid (BNA)) is a modified RNA nucleotide in which the ribose moiety is modified with an extra bridge connecting the 2′ oxygen and 4′ carbon. The bridge “locks” the ribose in the 3-endo conformation, which is often found in the A-form duplexes. This structure has been attributed to the increased stability against enzymatic degradation. Moreover the structure of LNA has improved specificity and affinity as a monomer or a constituent of an oligonucleotide such as a primer. LNA nucleotides can be mixed with DNA or RNA residues in the oligonucleotide, effectively hybridizing with DNA or RNA according to Watson-Crick base-pairing rules.

Without wishing to be bound by this theory the inventors believe that the LNA primers work in the context of the method of the invention since LNAs increase the affinity to the target nucleic acid. Hence, in all embodiments of the present invention as described herein it is possible to replace or replace in part the LNAs by other affinity-increasing modifications. Preferred examples of such other affinity affinity-increasing modifications are PNAs (Peptide Nucleic Acids, Biol Chem. August-September 1998; 379(8-9):1045-52.), ZNAs (Noir, R. et al. (2008) Oligonucleotide-oligospermine conjugates (Zip Nucleic Acids): a convenient means of finely tuning hybridization temperatures. J Am Chem Soc, 130, 13500-13505), 2′,4′-BNA(NC) (Nucleic Acids Symp Ser (Oxf). 2006; (50):195-6.) and RNA.

Hence, the present invention also relates, for example, to a method for the detection of a target nucleic acid sequence in a sample, wherein the method comprises: a) optionally contacting the sample with a lysis buffer under conditions wherein the sample is lysed, b) optionally heating the lysis buffer; c) subjecting the lysate as obtained after step a) and optionally after step b) or the sample to an isothermal amplification reaction at a temperature of 30 to 75° C., preferably 45 to 75°, more preferably 60 to 70° C., even more preferred about 65° C. with at least two primers specifically amplifying the target nucleic acid sequence, wherein the nucleotides of at least one primer comprise at least two affinity-increasing modifications, wherein the affinity-increasing modifications are preferably each independently selected from LNAs, PNAs, ZNAs, 2′,4′-BNA(NC) and RNA, which affinity-increasing modifications are not directly adjacent to each other within the nucleotides of the at least one primer, and wherein the isothermal amplification reaction comprises a DNA polymerase with reverse transcriptase activity and strand displacement activity and does not comprise a thermostable reverse transcriptase; and d) detecting the presence of the target nucleic acid sequence in the amplification product obtained after or during step d).

As already discussed, all isothermal amplification reactions have in common that they use a DNA polymerase with strand-displacement activity. The term strand displacement activity describes the ability of the DNA polymerase to displace downstream DNA encountered during synthesis.

The DNA polymerase to be used in accordance with the method of the invention not only has strand-displacement activity but also has reverse transcriptase activity. By the reverse transcriptase activity the conversion of RNA template molecules into a DNA double helix in catalyzed.

In the isothermal amplification reaction to be used in accordance with the method of the first aspect of invention no thermostable reverse transcriptase is used. The only reverse transcriptase activity that is present in the reaction is the reverse transcriptase activity of the DNA polymerase to be used. No other enzyme in the isothermal amplification reaction to be used in accordance with the method of the invention displays any reverse transcriptase activity. In particular, no separate thermostable reverse transcriptase is used.

Means for the detection of a target nucleic acid sequence in an amplification product are known in the art. Non-limiting examples are agarose gel and/or polyacrylamide gel electrophoresis, high-pressure liquid chromatography, electrochemiluminescence and direct sequencing. It is also possible to incorporate radioactive or nonradioactive labels directly into the amplified products. A variety of different nonradioactive labels are available for labeling DNA probes, including biotin, digoxigenin, horseradish peroxidase (HRP) and fluorescein. In particular, a barcoding system and method may be used to detect various different target nucleic acid sequences in multiplexed manner within the same sample. An example of barcoding system and method will be further explained herein below.

It is known from Jang et al. (1999), Gen Virol; 80(Pt 3):711-716 and the commercially available RT-PCR Quick Master Mix “One-step RT-PCR Master Mix” as obtainable from TOYOBO that the thermostable DNA polymerase derived from Thermus thermophilus (Tth) (e.g. strain HB8) can be used in a one-step RT-PCR including reverse transcription and PCR steps without the need to use a thermostable reverse transcriptase. The enzyme has a reverse transcriptase activity in addition to a 5′→3′ polymerase activity and a double strand specific 5′→3′ exonuclease activity in the presence of Mn²⁺ ions. In a RT-PCR the reverse transcriptase activity of the Tth DNA polymerase is sufficient, so that no extra reverse transcriptase activity is needed.

As can be taken from the appended examples the Bst polymerase is an example of a DNA polymerase with reverse transcriptase activity and strand displacement activity. The Bst polymerase was isolated from Bacillus stearothermophilus. The Bst polymerase features a similar 5′-3′ polymerase activity of E. coli, but other than the Tth polymerase lacks the 3′-5′ exonuclease activity. Bst polymerase is used in the art for isothermal amplification reactions.

The transcriptase activity of strand-displacing DNA polymerases is discussed in the prior art rather as a disadvantage. For example, Wang et al. (2017) Scientific Reports volume 7, Article number: 13928 teaches to remove RT activity from strand-displacing DNA polymerases in order to avoid non-specific amplification thoroughly.

The present inventors initially failed when trying to use the RT activity of the strand-displacing DNA polymerase Bst in an isothermal amplification reaction, in particular a RT-LAMP reaction. In contrast to the known one-step RT-PCR with the Tth DNA polymerase which does not need any extra reverse transcriptase activity, it turned out that the RT activity of the Bst polymerase alone was not sufficient in an RT-LAMP reaction. An amplification of the target nucleic acid sequence was only obtained in case in addition a thermostable reverse transcriptase was used as an additional enzyme in the reaction. This has in particular been exemplified for the diagnosis of SARS-CoV-2 infections and Influenza A infections.

It was then unexpectedly found that in case the outer primers (F3 and B3) in the RT-LAMP reaction are modified by LNAs, such that the LNAs are not found in a block in the primers but can only be found at every second nucleic acid position of the outer primers an amplification of the target nucleic acid is obtained in RT-LAMP reaction solely by the RT activity of the Bst polymerase and without a thermostable reverse transcriptase or any other enzyme displaying RT activity. In this connection the RT can be a carried out as a one-step RT-PCR, wherein cDNA synthesis and PCR are performed in a single reaction vessel in a common reaction buffer or as a two-step RT-PCR, wherein the cDNA is synthesized in one reaction, and an aliquot of the cDNA is then used for a subsequent PCR experiment (see Example 8).

This technical advantage of LNAs in isothermal amplification reactions is to the best knowledge of the inventors neither taught nor suggested by the prior art. Moreover, the avoidance of the need of a thermostable reverse transcriptase or any other additional enzyme other than the DNA polymerase displaying RT activity in an isothermal amplification reaction is a significant step towards sensitive, scalable, and multiplexed population-scale testing for the occurrence of a disease, in particular an infection in a population. This is because the most expensive enzyme in prior art isothermal amplification reactions is the thermostable reverse transcriptase. The avoidance of the need of this enzyme results in a significant cost reduction of population-scale testing. The novel method of the invention is also referred to herein as LNA-LAMP.

The present invention relates in a second aspect to a method for the detection of a target nucleic acid sequence in a sample, wherein the method comprises: a) optionally contacting the sample with a lysis buffer under conditions wherein the sample is lysed, wherein the lysis buffer comprises weak-acid-ion-exchange particles and/or granular activated carbon (GAC), b) optionally heating the Iysis buffer; c) subjecting the lysate as obtained after step a) and optionally after step b) or the sample to an isothermal amplification reaction at a temperature of 30 to 75° C., more preferably 45 to 75° C., more preferably 60 to 70° C., even more preferred about 65° C. with at least two primers specifically amplifying the target nucleic acid sequence; and d) detecting the presence of the target nucleic acid sequence in the amplification product obtained after or during step d).

The definitions and preferred options as discussed herein above in connection with the first aspect apply mutatis mutandis to the second aspect of the invention. In accordance with the second aspect in step (a) the lysis buffer comprises weak-acid-ion-exchange particles and/or granular activated carbon (GAC) but in the isothermal amplification reaction in step (c) only preferably and not necessarily at least one primer comprising at least two locked nucleic acids (LNAs) which are not directly adjacent to each other within the nucleotides of the at least one primer is used. The isothermal amplification reaction of the second aspect uses a DNA polymerase with strand displacement activity. In case at least one primer with LNAs is used the DNA polymerase preferably also displays reverse transcriptase activity and the isothermal amplification reaction does not comprise a thermostable reverse transcriptase or any other enzyme with RT activity other than the RT activity of the DNA polymerase.

Weak-acid-ion-exchange particles are commercially available and also methods for their preparation are available (for example, Zhou et al. (2005). J Chromatogr A., 1085(1):18-22). Weak-acid-ion-exchange particles are generally made of a resin or polymer and they act as a medium for ion exchange. The beads generally have a radius of 0.25-0.5 mm. The particles are typically porous, providing a large surface area on and inside them. The trapping of ions occurs along with the accompanying release of other ions, and thus the process is called ion exchange. There are multiple types of particles. Most commercial particles are made of polystyrene sulfonate. The weak-acid-ion-exchange particles are preferably ResinTech WACMP as used in the examples.

ResinTech WACMP is a hydrogen form macroporous weak acid cation resin. WACMP is an exceptionally high capacity resin and can be regenerated at close to 100% acid efficiency. ResinTech WACMP has low swelling and high physical strength when compared to gel weak acid cation resins. ResinTech WACMP has the following properties:

| | |
|---|---|
| Polymer Structure | Acrylic/DVB |
| Polymer Type | Macroporous |
| Functional Group | Carboxylic Acid |
| Physical Form | Spherical Beads |
| Ionic Form (as shipped) | Hydrogen |
| Total capacity | >4 meq/mL (H Form)/>2.2 meq/mL (Na Form) |
| Water Retention | 43 to 60 percent (H Form)/43 to 60 percent |
| | (Na Form) |
| Approximate | 47 lbs/cu. ft. (H Form)/46 lbs/cu. ft. |
| Shipping Weight | (Na Form) |
| Swelling | 50 to 60 percent Na to H |
| Screen Size Distribution | 16 to 50 (U.S. Mesh) |
| Maximum Fines Content | 1 percent (less than 50 Mesh) |
| Minimum Sphericity | 93 percent |
| Uniformity Coefficient | 1.7 (Approximate) |
| Resin Color | White to tan |

Granular activated carbon (GAC) has a relatively larger particle size compared to powdered activated carbon and consequently, presents a smaller external surface. These carbons are suitable for adsorption of gases and vapors, because they diffuse rapidly. Granulated carbons are used for water treatment, deodorization and separation of components of flow systems and are also used in rapid mix basins. GAC can be either in granular or extruded form and herein preferably a granular form is used.

The GAC is preferably coconut shell-based GAC. Coconut shell-based GAC is predominantly microporous and is well-suited for organic chemical adsorption. Coconut shell-based GAC has the highest hardness compared to other types of CAG, which makes it the ideal CAG for water purification. The coconut shell-based GAC is preferably the commercial product ResinTech AGC-40-CS AW. ResinTech AGC-40-CS AW has the following properties:

| | |
|---|---|
| Type | Coconut Shell |
| Screen Size | <5% (+12 mesh)/<5% (−40 mesh) |
| Iodine Number | 950 |
| pH (potable water) | 5 to 8 (acid washed) |
| Conductivity | 500 uS/cm (acid washed) |
| Apparent Density | 0.68 (moist) |
| Shipping Weight | 38 lbs/cu. ft. (moist) |

The (coconut shell-based) GAC may be washed one or more times with water in order to remove impurities before the (coconut shell-based) GAC is added to the lysis buffer. Such (coconut shell-based) GAC is referred to herein as washed (coconut shell-based) GAC.

As is illustrated by the appended examples, the addition of weak-acid-ion-exchange particles and/or granular activated carbon (GAC) into the lysis buffer removes impurities from the example which in turn increases the sensitivity and reliability of the method for the detection of a target nucleic acid sequence in a sample.

The present invention relates in a third aspect to a method for the detection of a target nucleic acid sequence in a sample, wherein the method comprises: a) optionally contacting the sample with a lysis buffer under conditions wherein the sample is lysed, wherein the lysis buffer is an aqueous lysis buffer comprising 100 to 1000 mM pH 8.0 to pH 9.0, preferably about Tris pH 8.5, 1 to 6 M Trimethylglycin, and 1 to 10 u/ml Proteinase, b) optionally heating the lysis buffer; c) subjecting the lysate as obtained after step a) and optionally after step b) or the sample to an isothermal amplification reaction at a temperature of 30 to 75° C., preferably 45 to 75° C., more preferably 60 to 70° C., even more preferred about 65° C. with at least two primers specifically amplifying the target nucleic acid sequence; d) detecting the presence of a target nucleic acid sequence in the amplification product obtained after or during step d).

The definitions and preferred options as discussed herein above in connection with the first and second aspect apply mutatis mutandis to the third aspect of the invention. In accordance with the third aspect in step (a) the lysis buffer comprises 100 to 1000 mM Tris pH 8.0 to pH 9.0, preferably about pH 8.5, 1 to 6 M Trimethylglycin, and 1 to 10 u/ml Proteinase. In step (a) only preferably and not necessarily weak-acid-ion-exchange particles and/or granular activated carbon (GAC) are used and also in the isothermal amplification reaction in step (c) only preferably and not necessarily at least one primer comprising at least two locked nucleic acids (LNAs) which are not directly adjacent to each other within the nucleotides of the at least one primer is used. The isothermal amplification reaction of the second aspect uses a DNA polymerase with strand displacement activity. In case at least one primer with LNAs is used the DNA polymerase preferably also displays reverse transcriptase activity and the isothermal amplification reaction does not comprise a thermostable reverse transcriptase or any other enzyme with RT activity other than the RT activity of the DNA polymerase.

The term “aqueous lysis buffer” stipulates that the solvent of the lysis buffer is water. Hence, within the lysis buffer as used in connection with the third aspect of the invention Tris pH 8.0 to pH 9.0, preferably about pH 8.5, Trimethylglycin, and the Proteinase are dissolved in water.

Tris pH 8.0 to pH 9.0, preferably about pH 8.5 (tris-(hydroxymethyl)-aminomethane; formula (HOCH₂)₃CNH₂) is a buffer system that maintains the pH close to pH 8.0 to pH 9.0, preferably about 8.5. A buffer comprises a substance which by its presence in solution increases the amount of acid or alkali that must be added to cause unit change in pH. Buffers are thus very important components in experiments designed to study biological reactions by maintaining a constant concentration of hydrogen ions within the physiological range. The 100 to 1000 mM Tris pH 8.0 to pH 9.0, preferably about pH 8.3 are preferably 200 to 400 mM Tris pH 8.0 to pH 9.0, preferably about pH 8.5 and most preferably about 300 mM.

Trimethylglycin (or TMG or beatin or glycine betaine; UIPAC name N,N,N-Trimethylammonioacetat) is an N-methylated amino acid. It is a zwitterion as the molecule contains both a quaternary ammonium group and a carboxyl group. The carboxyl group will be partially protonated in an aqueous solution below pH 4. Trimethylglycine can act as an adjuvant of the polymerase chain reaction (PCR) process, and other DNA polymerase-based assays, such as DNA sequencing. By an unknown mechanism, it aids in the prevention of secondary structures in the DNA molecules, and prevents problems associated with the amplification and sequencing of GC-rich regions. Trimethylglycine makes guanosine and cytidine (strong binders) behave with thermodynamics similar to those of thymidine and adenosine (weak binders). The 1 to 6 M concentration of Trimethylglycin are preferably 1.5 to 5M, more preferably 2 to 3M and most preferably about 2.5M.

A protease (also called a peptidase or proteinase) is an enzyme that catalyzes (increases the rate of) proteolysis, the breakdown of proteins into smaller (poly)peptides or single amino acids. The protease is preferably proteinase K (EC 3.4.21.64, protease K, endopeptidase K, Tritirachium alkaline proteinase, Tritirachium album serine proteinase, Tritirachium album proteinase K). Proteinase K is a broad-spectrum serine protease. Proteinase K is capable to digest hair (keratin), hence, the name “Proteinase K”. The predominant site of cleavage is the peptide bond adjacent to the carboxyl group of aliphatic and aromatic amino acids with blocked alpha amino groups. It is commonly used for its broad specificity. This enzyme has a molecular weight of 28.9 kDa. The 1 to 10 u/ml Proteinase are preferably 1.5 to 5 u/ml Proteinase, more preferably 1.8 to 3.2 u/ml Proteinase and most preferably about 2.7 u/ml Proteinase.

As can be taken from the appended examples, the use of the particular lysis buffer in accordance with the third aspect of the invention results in a highly sensitive and reliable method for the detection of a target nucleic acid sequence in a sample. In the examples a number of different lysis buffers were tested that can be distinguished from each other by the use of another detergent (Trition X-100 or Tween-20) instead of Trimethylglycin. The buffer with Trimethylglycin was clearly the best with respect to the detection sensitivity. In addition, the lysis buffer in accordance with the third aspect of the invention only comprises water, Tris pH 8.0 to pH 9.0, preferably about pH 8.5, Trimethylglycin, and a Proteinase. Thus, the lysis buffer can be easily produced and only comprises inexpensive ingredients. Altogether the lysis buffer is ideal for reducing the costs of population-scale testing.

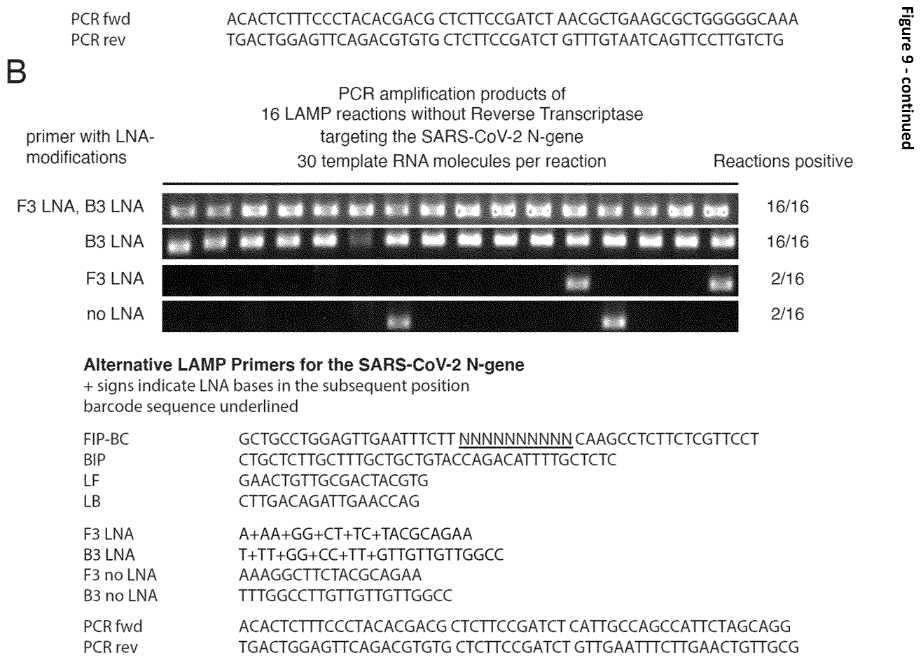

In accordance with a preferred embodiment of the first, second and third aspect of the invention in step c) the isothermal amplification reaction with at least two primers specifically amplifying the target nucleic acid sequence is a reverse transcriptase loop-mediated isothermal amplification (RT-LAMP) reaction with at least four primers specifically amplifying the target nucleic acid sequence, wherein two primers are outer primers and at least two primers are inner primers, and wherein the at least one primer comprising the at least two locked nucleic acids (LNAs), if present, is one (F3 or B3 primer), preferably both of the outer primers (F3 and B3 primer). The one primer of the outer primers is preferably the B3 primer. The F3 primer is not required to comprise the at least two LNAs (see FIG. 9 and Example 10). The other primers of the RT-LAMP; i.e. the inner primers and, if present, the loop primers do not comprise LNAs (i.e. the FIP and BIP primer, and, if present, the LB and LF primers).

As mentioned above, reverse transcriptase loop-mediated isothermal amplification (RT-LAMP) is a type of an isothermal amplification reaction. Loop-mediated isothermal amplification (LAMP) uses 4-6 primers recognizing 6-8 distinct regions of target DNA for a highly specific amplification reaction. A strand-displacing DNA polymerase initiates synthesis and 2 specially designed primers form “loop” structures to facilitate subsequent rounds of amplification through extension on the loops and additional annealing of primers. DNA products are very long (>20 kb) and formed from numerous repeats of the short (80-250 bp) target sequence, connected with single-stranded loop regions in long concatamers. These products are not typically appropriate for downstream manipulation, but target amplification is so extensive that numerous modes of detection are possible. Real-time fluorescence detection using intercalators or probes, lateral flow and agarose gel detection are all directly compatible with LAMP reactions. Instrumentation for LAMP typically requires consistent heating to the desired reaction temperature and, where needed, real-time fluorescence for quantitative measurements.

The design of LAMP primers can be done with the help of software tools. As discussed, an RT-LAMP is characterized by the use of at least 4 different primers specifically designed to recognize 6 distinct regions of the target gene. The four primers being used are designated in the prior art follows:
Forward Inner Primer (FIP)
Forward Outer Primer (FOP) (also called F3 Primer)
Backward Inner Primer (BIP)
Backward Outer Primer (BOP) (also called B3 Primer)
In the case of six primers a backward and forward loop primer (LB and LF primer) are used in addition. forward loop primers; LB, backward loop primers.

Hence, the LAMP primers comprise at least an outer primer pair and an inner primer pair.

The stages of LAMP after the reverse transcription of the target RNA into target DNA may be summarizes as follows: 1. F2 region of FIP hybridizes to F2c region of the target DNA and initiates complementary strand synthesis. 2. Outer primer F3 hybridizes to the F3c region of the target DNA and extends, displacing the FIP linked complementary strand. This displaced strand forms a loop at the 5′ end. 3. This single stranded DNA with a loop at the 5′ end serves as a template for BIP. 82 hybridizes to 82c region of the template DNA. DNA synthesis is now initiated leading to the formation of a complementary strand and opening of the 5′ end loop. 4. The outer primer 83 hybridizes to B3c region of the target DNA and extends, displacing the BIP linked complementary strand. This results in the formation of a dumbbell shaped DNA. 5. The nucleotides are added to the 3′ end of F1 by DNA polymerase, which extends and opens up the loop at the 5′ end. The dumbbell shaped DNA now gets converted to a stem loop structure. This structure serves as an initiator for LAMP cycling, which is the second stage of the LAMP reaction. 6. To initiate LAMP cycling, the FIP hybridizes to the loop of the stem-loop DNA structure. Strand synthesis is initiated here. As the FIP hybridizes to the loop, the F1 strand is displaced and forms a new loop at the 3′ end. 7. Nucleotides are added to the 3′ end of B1. The extension takes place displacing the FIP strand. This displaced strand again forms a dumbbell shaped DNA. Subsequent self-primed strand displacement DNA synthesis yields one complementary structure of the original stem loop DNA and one gap repaired stem loop DNA. 8. Both these products then serve as template for a BIP primed strand displacement reaction in the subsequent cycles. Thus, a LAMP target sequence is amplified 13-fold every half cycle. The final products obtained are a mixture of stem loop DNA with various stem lengths and various cauliflower like structures with multiple loops. The structures are formed by annealing between alternatively inverted repeats of the target sequence in the same strand. The optional loop primers LF and LB are located between F2 and F1 or 81 and 82. They are designed to anneal at the loop structure of the amplicons and accelerate and enhance the sensitivity of the RT-LAMP.

In accordance with a more preferred embodiment of the first, second and third aspect of the invention the RT-LAMP reaction is a LAMP-Seq reaction.

LAMP-Seq is a relatively novel RT-LAMP reaction that has been published by Schmid-Burgk et al. (2020) (bioRxiv preprint doi: https://doi.org/10.1101/2020.04.06.025635). LAMP-Seq is a barcoded RT-LAMP method that is highly scalable. Individual samples are stabilized, inactivated, and amplified in three isothermal heat steps, generating barcoded amplicons that can be pooled and analyzed en masse by sequencing. Using unique barcode combinations per sample from a compressed barcode space enables extensive pooling, potentially further reducing cost and simplifying logistics.

In LAMP-Seq the barcode sequences are generally inserted into the forward inner primer (FIP), which enables generation of barcoded palindromic amplification products. The barcode is preferably a 5 to 15 nucleotides, more preferably a 10 nucleotides barcode with a GC content of 30%-70% and lacking homopolymer repeats of four or more nucleotides. It was found that the use of a barcode does not affect LAMP sensitivity, product amounts, or downstream PCR amplification.

In accordance with a preferred embodiment of the first, second and third aspect of the invention the DNA polymerase with reverse transcriptase activity and strand displacement activity is selected from (i) a Bacillus stearothermophilus (Bst) DNA Polymerase I, a large fragment of Bst DNA Polymerase I, or a homolog thereof retaining 5′→3′ DNA polymerase activity, reverse transcriptase activity and strong strand displacement activity of the large fragment of Bst DNA Polymerase I, and/or
(ii) the Bsu DNA Polymerase, Large Fragment.

A Bst DNA Polymerase I has already been mentioned herein above. Bst DNA Polymerase I was isolated from Bacillus stearothermophilus. The enzyme displays polymerase activity and strand displacement activity but lacks 3′-5′ exonuclease activity. It also has reverse transcription activity. The Bst DNA Polymerase I preferably comprises SEQ ID NO: 1 or a sequence being with increased preference at least 90%, at least 91%, at least 92%, at least 93%, at least 94%, at least 95%, at least 96%, at least 97%, at least 98% and at least 99% identical to SEQ ID NO: 1. SEQ ID NO: 1 is also known as the Bst-LF-Idaho polymerase and was published by Kiefer, J. R. et al. Crystal structure of a thermostable Bacillus DNA polymerase I large fragment at 2.1 A resolution. Structure 5, 95-108 1997. The Bst-LF-Idaho polymerase is the large fragment of Bst DNA Polymerase I as used in the examples herein below. It is noted that the Bst-LF-Idaho polymerase sequence as shown in the Example (SEQ ID NO: 30) comprises at the N-terminus (first 19 amino acids) a poly-His purification tag which is not part of SEQ ID NO: 1.

A large fragment of Bst DNA Polymerase I comprises 67 kDa and retains the polymerase activity, strand displacement activity and reverse transcription activity of a full-length Bst DNA Polymerase I. Also the large fragment of the Bst DNA Polymerase I preferably comprises or consists of the large fragment of the Bst DNA Polymerase I of SEQ ID NO: 1 or a sequence being with increased preference at least 90%, at least 91%, at least 92%, at least 93%, at least 94%, at least 95%, at least 96%, at least 97%, at least 98% and at least 99% identical to SEQ ID NO: 1. Among the options of the above preferred embodiment of the first, second and third aspect of the invention the DNA polymerase with reverse transcriptase activity and strand displacement activity is preferably a large fragment of Bst DNA Polymerase I and is most preferably a large fragment of Bst DNA Polymerase I comprising or consisting of the large fragment of the Bst DNA Polymerase I of SEQ ID NO: 1 or a sequence being with increased preference at least 90%, at least 91%, at least 92%, at least 93%, at least 94%, at least 95%, at least 96%, at least 97%, at least 98% and at least 99% identical to SEQ ID NO: 1.

Homologs of Bst DNA Polymerase I retaining 5′→3′ DNA polymerase activity and also retaining the strand displacement activity and reverse transcription activity are known in the art. For example, Bst 2.0 DNA Polymerase displays improved amplification speed, yield and salt tolerance as compared to Bst DNA Polymerase I. Bst 3.0 DNA Polymerase demonstrates robust performance even in high concentrations of amplification inhibitors and features significantly increased reverse transcriptase activity as compared to Bst DNA Polymerase I. A further example is the exo-variant of a Bst DNA Polymerase I, which has been modified to have no exonuclease activity.

The Bsu DNA Polymerase, Large Fragment is a further example of a DNA polymerase with at least some strand displacement activity and reverse transcription activity.

The Bsu DNA Polymerase, Large Fragment retains the 5′→3′ polymerase activity of the Bacillus subtilis DNA polymerase I, but lacks the 5′→3′ exonuclease domain. This large fragment naturally lacks 3′→5′ exonuclease activity.

In accordance with a preferred embodiment of the first and third aspect of the invention the lysis buffer comprises weak-acid-ion-exchange particles and/or granular activated carbon (GAC).

In accordance with a more preferred embodiment of the first, second and third aspect of the invention the GAC are coconut shell-based GAC and preferably washed coconut shell-based GAC.

In accordance with a further more preferred embodiment of the first, second and third aspect of the invention per 100 μl of lysis buffer 10-60 milligram, preferably 20-50 milligram and most preferably about 35 milligram of weak-acid-ion-exchange particles and/or GAC are comprised in the lysis buffer.

Weak-acid-ion-exchange particles and/or granular activated carbon (GAC), including coconut shell-based GAC and washed coconut shell-based GAC have been described herein above in connection with the second aspect of the invention. The definitions and preferred options as discussed herein above in connection with the second aspect apply mutatis mutandis to the above preferred embodiment and more preferred embodiment of the first and third aspect of the invention.

In accordance with a preferred embodiment of the first and second aspect of the invention the lysis buffer is an aqueous lysis buffer comprising 100 to 1000 mM Tris pH 8.0 to pH 9.0, preferably about pH 8.5, 1 to 6 M Trimethylglycin, and 1 to 10 u/ml Proteinase.

This aqueous lysis buffer including preferred embodiments thereof has been described in connection with the third aspect of the invention. The definitions and preferred options as discussed herein above in connection with the third aspect apply mutatis mutandis to the above preferred embodiment of the first and second aspect of the invention.

In accordance with another preferred embodiment of the first, second and third aspect of the invention the sample is a swab sample, preferably an oropharyngeal, nasopharyngeal, anal, buccal, skin or mid-nasal swab sample.

The above samples are preferred examples of samples that are used for population-scale testing for the occurrence of a disease, preferably an infection in a population because these samples can be obtained by non-invasive and non-surgical methods. These samples can also be obtained by non-medically qualified people and in particular by a subject in self-test at home.

As is illustrated by Example 9, the swab samples can also be analyzed in an automation-compatible sample format and should ideally be transferred to such a format from the earliest possible step of the testing procedure, i.e. directly after the sample has been obtained.

The automation-compatible sample format is preferably a well plate, such as a 6, 12, 24, 48, 96, 384, 1536 or 3456 well plate (preferably a 96-well plate). The well plate is preferably a pre-filled with lysis buffer and sealed with pierceable lid, preferably with a pierceable aluminium, paper, plastic or composite foil, wherein the composite is made from two or all three of aluminium, paper and plastic.

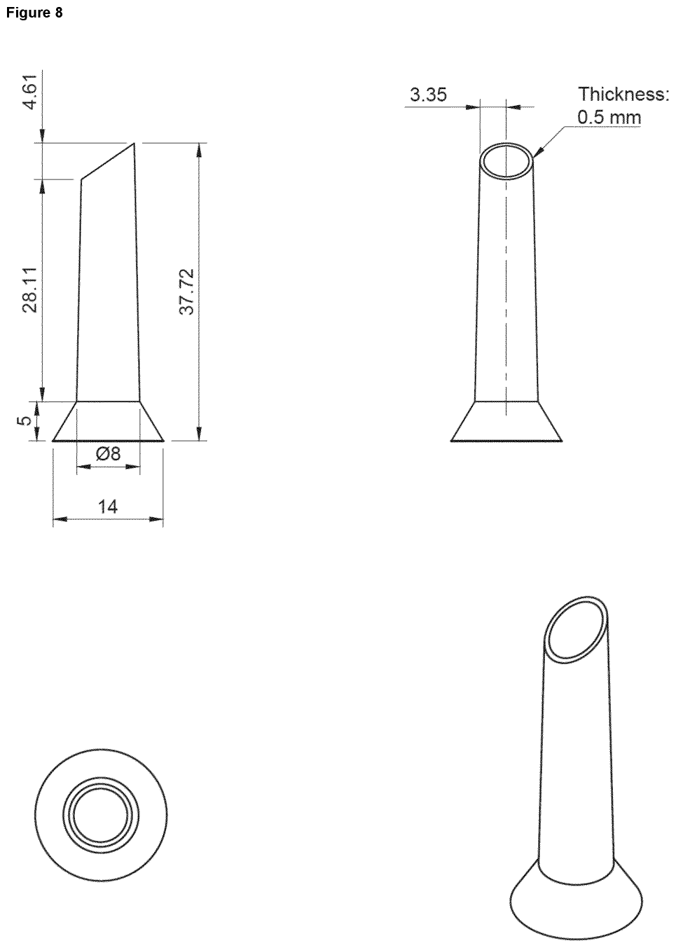

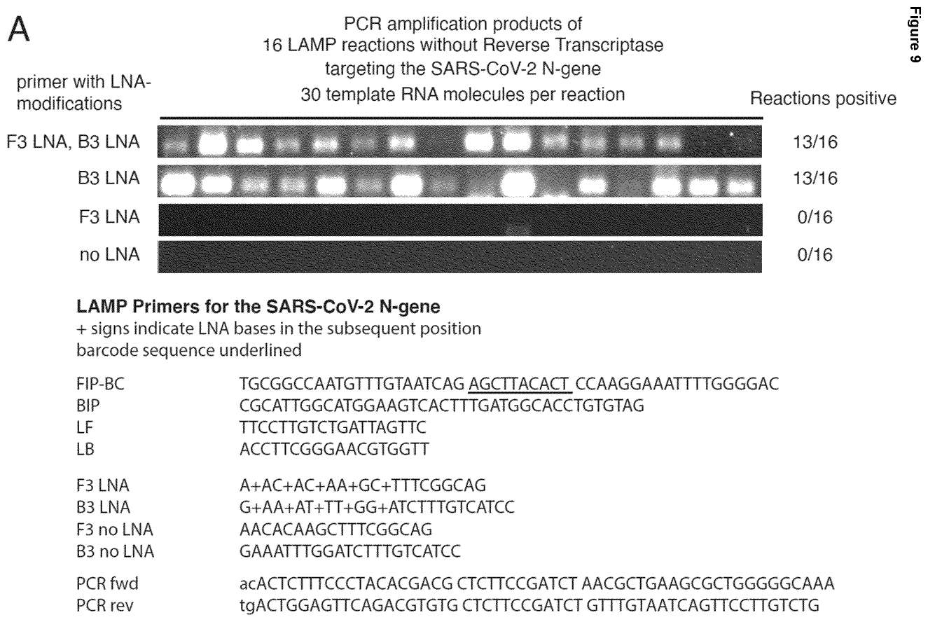

The swab sample is preferably transferred into a well of the well-plate with a piercing funnel device with piercing funnel device. The piercing funnel device has an open distal end which allows piercing an individual well of the plate. The open distal end is preferably formed like the end of syringe injection needle. The piercing funnel device furthermore has an open proximal end and a tube between the proximal end and the distal end which enable the insertion of an inoculated swab through the piercing funnel device into a well of the plate. The open proximal end is preferably formed like the end of a funnel in order facilitate the insertion of the swap. An example of such a piercing funnel device is shown in FIG. 8. Accordingly, the piercing funnel device preferably has a maximum height of about 37.72 mm and/or a minimum height of about 28.11 mm. The bevel forming the syringe injection needle-like end is preferably about 4.61 mm of the maximum height. The funnel-like end is preferably about 5.00 mm of the maximum height. The maximum diameter of the tube at the distal end is preferably about 3.35 mm and/or the maximum diameter of the tube at the proximal end is preferably about 8.00 mm. The wall of the tube preferably has a thickness of about 0.5 mm. The term “about” is preferably ±20% and more preferably ±10%. The material of the piercing funnel device is preferably plastic.

The use of such a piercing funnel device advantageously ensures a fast, safe, and convenient way to extract sample material from human swabs into lysis buffer, while minimizing cross-contamination of neighbouring wells or other parts of the working environment.

After the swab has been inserted through the piercing funnel device into a well of the plate optionally agitating in the lysis buffer can be performed for a defined time, such as about 1 min, about 30 see or about 10 sec.

The swab and funnel may be removed together from the well by retracting the wet portion of the swab.

Lastly, for storage until further analysis the well may be closed, e.g., by using a silicone plug or plastic cap.

In view of the above, the present invention is also directed to a piercing funnel device as described herein above as a product. In addition, the present invention is directed to a method for adding a swab sample into a well of a well plate comprising (a) piercing a selected well of a well plate being pre-filled with lysis buffer and sealed with pierceable lid (preferably with a pierceable aluminium, paper, plastic or composite foil, wherein the composite is made from two or all three of aluminium, paper and plastic) with the piercing funnel device, and (b) inserting the swab sample (preferably a swap sample as described herein above) through the piercing funnel device, and optionally (c) removing the swab sample and/or the piercing funnel device, and/or (d) closing the well, preferably with a plug or cap, such as a silicone plug or plastic cap.

In accordance with a related preferred embodiment of the first, second and third aspect of the sample is or comprises a body fluid, wherein the body fluid is preferably saliva, sputum, feces, urine, blood, serum, plasma, semen, vaginal fluid, mucus, tears, and milk.

The detection of the occurrence of a disease, preferably an infection in a subject is often based on a body fluid sample that has been obtained from the subject. Non-limiting but preferred examples of body fluids are listed in the above preferred embodiment.

In accordance with a further preferred embodiment of the first, second and third aspect in step b) the lysis buffer is heated to 60 to 100° C., preferably to about 95° C.

Heating the lysis buffer to 60 to 100° C., preferably to about 95° C. ensures the efficient inactivation of enzymes in the lysis buffer, such as Proteinase K. By the heating also the activity of unwanted enzymes in the sample, such as DNases or RNases may be inactivated or at least their activity might be reduced.

Yet further, the heat treatment helps to denature protein in general which may increase the accessibility to the target RNA in step (c) of the methods of the invention.

The time of the hearting step is preferably between 3 and 30 min, more preferably between 5 and 20 min and most preferably about 10 min, wherein “about” is preferably ±20% and more preferably ±10%.

In accordance with another preferred embodiment of the first, second and third aspect the sample is transferred from the lysis buffer to the amplification reaction by the same tool that was used to collect the sample.

The use of the same tool for the step of obtaining the sample (such as a swab sample, preferably an oropharyngeal, nasopharyngeal, anal, buccal, skin or mid-nasal swab sample) and the step of the transfer from the lysis buffer to the amplification reaction offers the advantages of minimizing the risk of contamination of the sample.

In accordance with a more preferred embodiment of the first, second and third aspect the tool is a swab, brush, toothpick, plastic stick, or spoon.

A swab, brush, toothpick, plastic stick, or spoon are non-limiting examples of easily accessible tools that are used in the art in order to obtain samples, such as a(n) oropharyngeal, nasopharyngeal, anal, buccal, skin or mid-nasal sample.

In accordance with a preferred embodiment of the first, second and third aspect the target nucleic acid sequence is a pathogenic nucleic acid sequence, preferably a viral nucleic acid sequence.

A pathogenic nucleic acid sequence is any nucleic acid sequence the presence of which in the sample is indicative for the presence of a disease, preferably an infection in the subject from whom the sample was taken. In the preferred case of a viral nucleic acid sequence the disease is a viral infection.

In accordance with a more preferred embodiment of the first, second and third aspect the viral nucleic acid sequence is a betacoronavirus nucleic acid sequence, influenza nucleic acid sequence or a RSV nucleic acid sequence.

A betacoronavirus, an influenza or a RSV are examples of viruses that cause disease in human subjects, in particular COVID-19, the Flu and rabies, respectively. The betacoronavirus and influenza caused and can cause a pandemic which requires population-scale testing for the occurrence of viral infections in a population.

In accordance with an even more preferred embodiment of the first, second and third aspect the betacoronavirus is preferably selected from SARS-CoV-2, MERS-CoV, SARS-CoV-1, OC43, and HKU1, and is most preferably SARS-CoV-2.

SARS-CoV-2, MERS-CoV, SARS-CoV-1, OC43, and HKU1 are known betacoronaviruses that are pathogenic for humans.

In accordance with a preferred embodiment of the first, second and third aspect the method comprises in step (c) at least two primer pairs comprising an outer primer pair and an inner primer pair, wherein the nucleotides of each of the primers of the outer primer pair comprise one or more LNAs.

As discussed herein above, the isothermal amplification reaction of the methods of the invention may comprise LAMP or related isothermal amplification methods. In such methods an outer primer pair and an inner primer pair can be used. In case the nucleotides of each of the primers of the outer primer pair comprise one or more LNAs or one of the preferred LNAs patterns as discussed herein above in connection with the first aspect of the invention, the isothermal amplification reaction can employ a DNA polymerase with reverse transcriptase activity and strand displacement activity and does not comprise a thermostable reverse transcriptase or any other enzyme with RT activity other than the RT activity of DNA polymerase.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

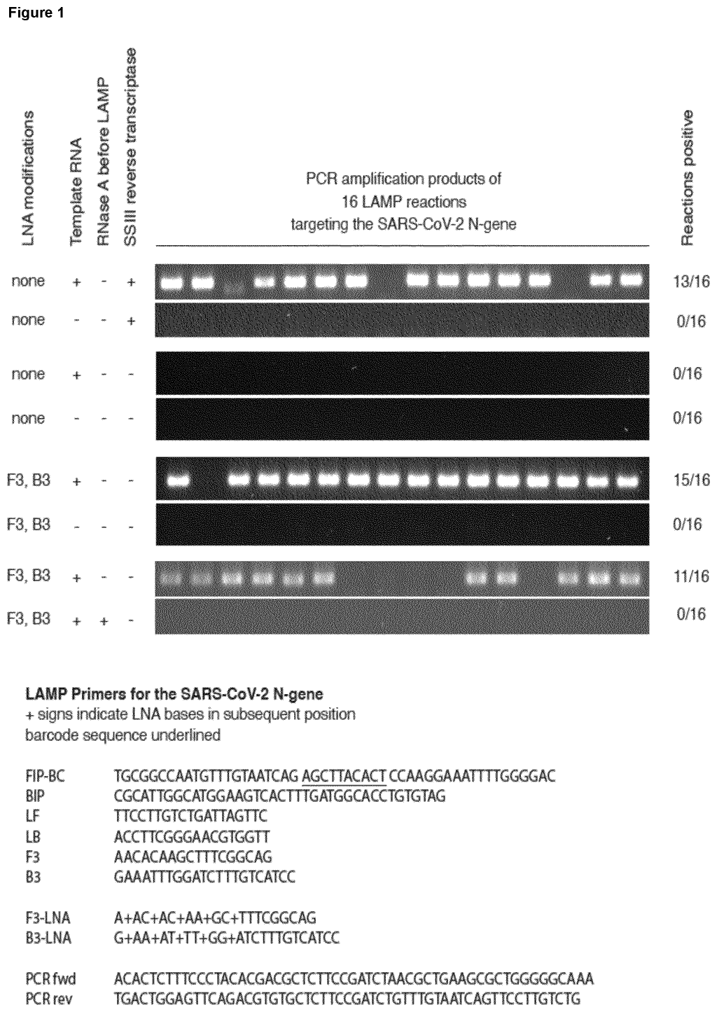

FIG. 1|Efficient LNA-LAMP without a reverse transcriptase. Shown are agarose gels of PCR amplification products of 16 RT-LAMP reactions per condition targeting SARS-CoV-2 N-gene RNA. Reactions were performed with or without template RNA (approximately 20 molecules per reaction), with or without RNase-A digestion of template RNA, with or without reverse transcriptase, and with or without LNA modifications in the F3 and B3 primers (sequences detailed in protocols section), as indicated on the left side of the panel. Numbers of reactions interpreted as positive are indicated on the right side of the gel.

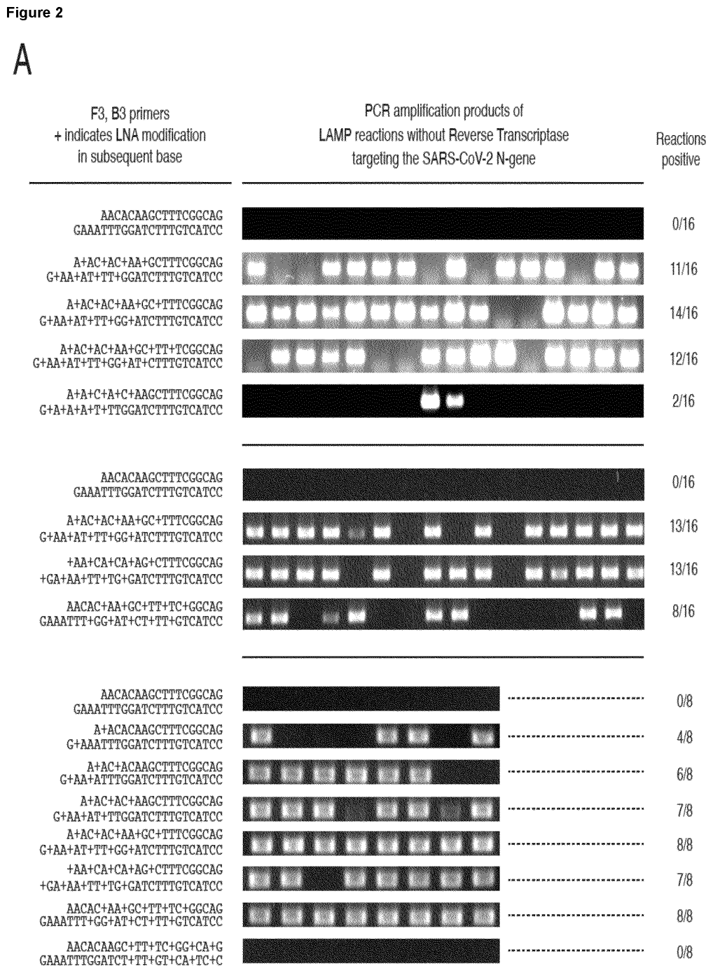

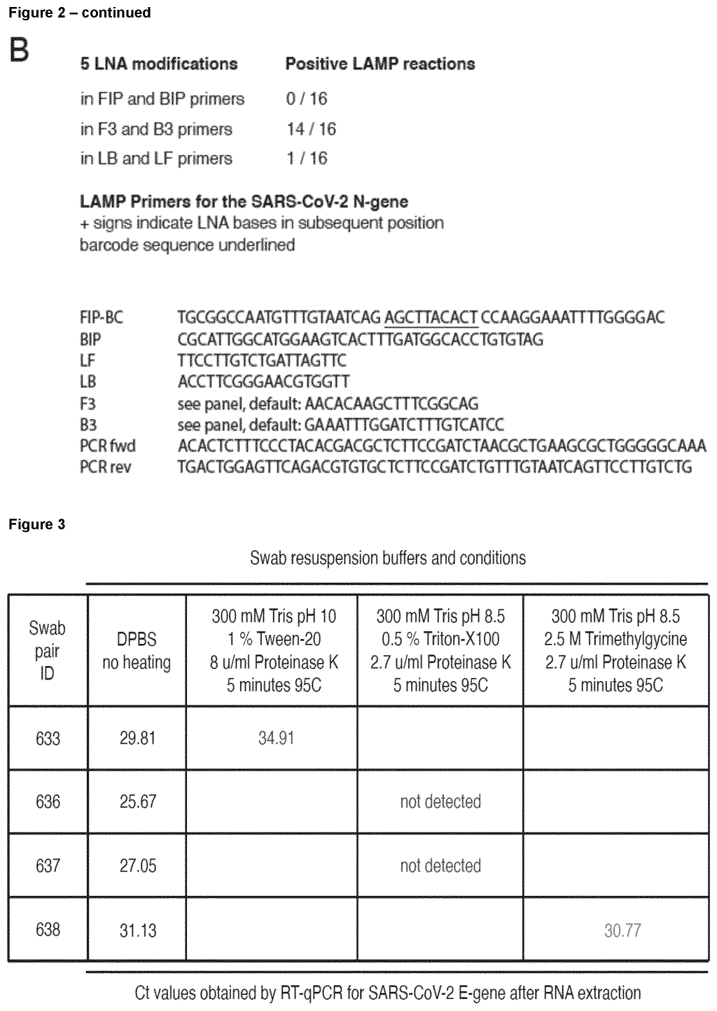

FIG. 2|Optimization of LNA modification patterns in RT-LAMP. (A) Shown are agarose gels of PCR amplification products of 8 or 16 RT-LAMP reactions per condition without reverse transcriptase, targeting SARS-CoV-2-N-gene RNA (approximately 20 molecules per reaction). Reactions were performed without or with LNA modifications in the F3 and B3 LAMP primers, as indicated on the left side of the panel (other primer sequences are provided in the protocols section). ‘+’ signs indicate LNA modifications in the subsequent base. Numbers of reactions interpreted as positive are indicated on the right side of the gel. (B) Shown are numbers of positive PCR amplification products of 16 RT-LAMP reactions per condition with reverse transcriptase, targeting SARS-CoV-2-N-gene RNA, with LNA modifications present in two out of six LAMP primers, as indicated in the left column.

FIG. 3|Lysis buffers and conditions suitable for extraction-free LNA-LAMP testing. Shown are the Threshold-Cycle (Ct) values obtained by RT-qPCR detecting the SARS-CoV-2 E-gene after parallel RNA extraction using the Swab resuspension buffers and conditions indicated in the first row of the table as compared to the standard clinical procedure of swab resuspension in DPBS at room temperature without heating (second column). Only the rightmost lysis condition yielded a similar amount of RNA after heating as compared to the no-heating condition.

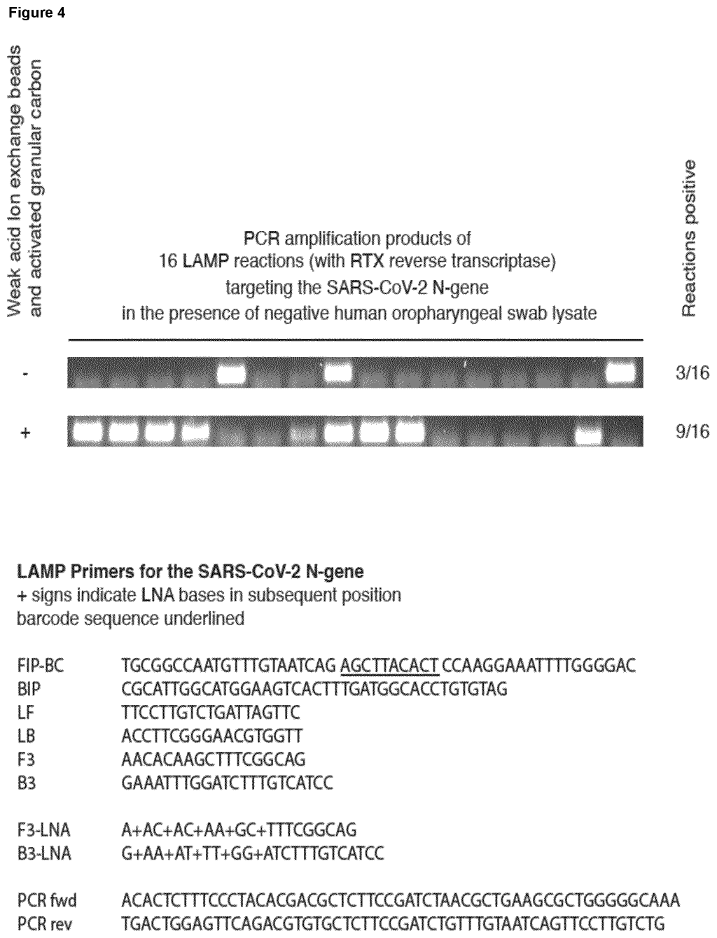

FIG. 4|Resin-assisted sensitive detection of viral RNA from unpurified human samples. Shown are agarose gels of PCR amplification products of 16 RT-LAMP reactions per condition with RTX reverse transcriptase, targeting SARS-CoV-2 N-gene RNA (approximately 20 template RNA molecules per reaction +human oropharyngeal swab lysate). Reactions were performed after swab lysates were optionally pre-incubated with weak acid Ion exchange beads and activated granular carbon, as indicated on the left side of the panel. Numbers of reactions interpreted as positive are indicated on the right side of the gel.

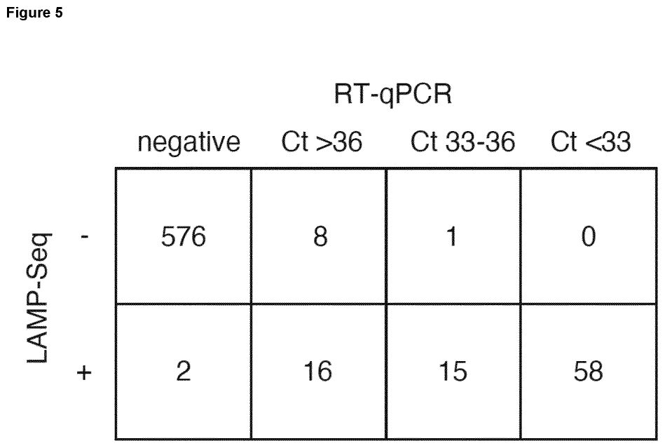

FIG. 5|Performance of purification-free LNA-LAMP against a standard clinical extraction-RT-qPCR pipeline. Shown are the qualitative results of 676 human oropharyngeal swab samples resuspended and heated in best lysis buffer from FIG. 3, and subjected to LNA-LAMP in four replicates per sample without purification, and analyzed by sequencing (LAMP-Seq). In parallel, lysates were analyzed by a clinically approved diagnostics pipeline consisting of RNA-extraction and RT-qPCR. Numbers indicate the total number of samples in each class of agreement between the two methods. LAMP-Seq samples with at least two out of four replicates called positive by sequencing were counted as positive.

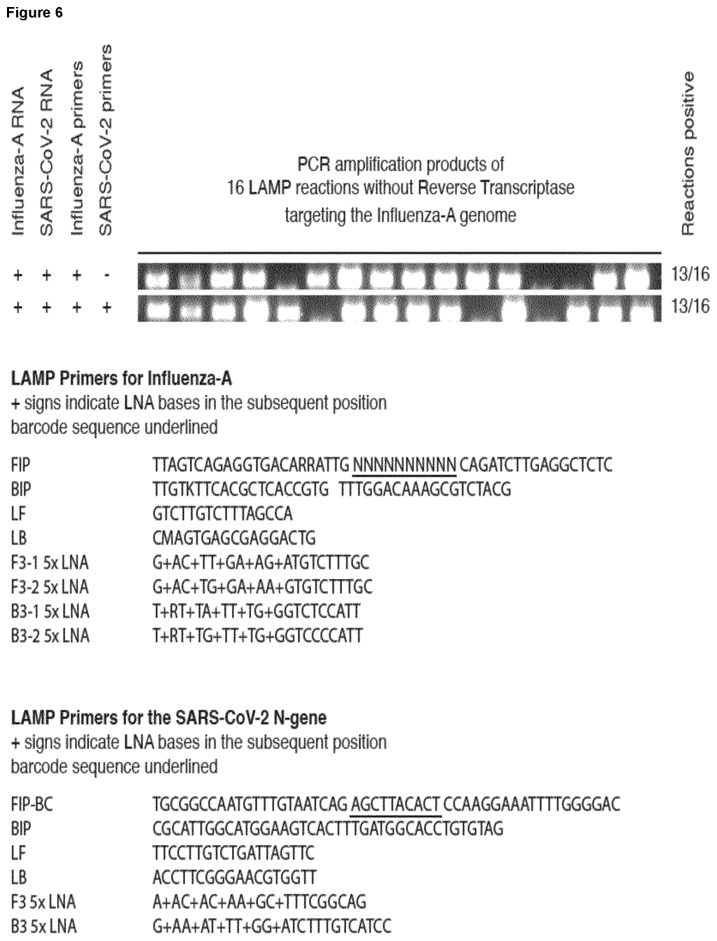

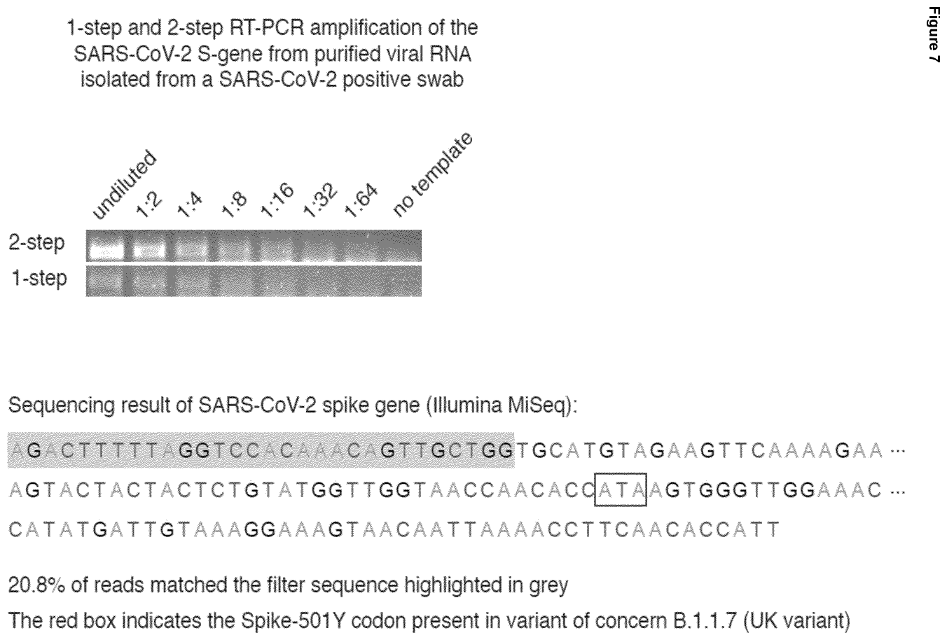

FIG. 6|Evaluation of LNA-LAMP-mediated detection of Influenza-A genomic RNA, optionally performed simultaneously with SARS-CoV-2 detection in a single reaction. Shown are agarose gels of PCR amplification products of 16 RT-LAMP reactions per condition without reverse transcriptase, targeting Influenza-A virus genomic RNA (approximately 20 template RNA molecules per reaction). Reactions were performed either in the presence or absence of LNA-LAMP primers targeting the SARS-CoV-2 N-gene as indicated. All reactions also contained SARS-CoV-2 N-gene RNA (approximately 20 molecules per reaction). F3 and B3 primers contained LNA modifications as detailed in the protocols section. Numbers of reactions interpreted as positive are indicated on the right side of the gel.

FIG. 7|Targeted RNA sequencing of the SARS-CoV-2 S-gene using LNA-assisted reverse transcription with a Bst polymerase using a 1-step or 2-step RT-PCR protocol. Top panel, SARS-CoV-2 genomic RNA purified from a positive human oropharyngeal swab sample was amplified using a 1-step or 2-step LNA-assisted RT-PCR protocol. Shown are agarose gels of amplification products of the expected size. Bottom panel, consensus sequencing result of SARS-CoV-2 S-gene amplification products using a 2-step LNA-assisted RT-PCR protocol. Products were sequenced on an Illumina MiSeq™ device and filtered for the presence of the sequence indicated in grey. 20.8% of raw sequencing reads passed the indicated filter. The sequencing result revealed the presence of a variant-of-concern (B.1.1.7, UK variant), with the defining codon 501 indicated by a red box.

FIG. 8|Piercing funnel device for fast and contamination-free sampling in a robotics-compatible format. Shown is an exemplary design of a piercing funnel device in front, top, and two 3D perspectives. The device is used to pierce open a well of a sample acquisition plate pre-filled with lysis buffer, and pre-sealed with a pierceable aluminum foil. After piercing, the device is used to insert a swab safely into the well, incubating for a defined amount of time, and removing it safely while avoiding contamination of neighboring wells.

FIG. 9|Determination of required LNA-modified primers in RT-LAMP without using a reverse transcriptase for two independent templates. (A) Shown are agarose gels of PCR amplification products of 16 RT-LAMP reactions per condition without reverse transcriptase, targeting SARS-CoV-2-N-gene RNA (30 template molecules per reaction). Reactions were performed without or with LNA modifications in the F3 and/or B3 LAMP primers, as indicated on the left side of the panel. ‘+’ signs indicate LNA modifications in the subsequent base. All primer sequences are indicated below. Numbers of reactions interpreted as positive are indicated on the right side of the gel. (B) Shown are agarose gels of PCR amplification products of 16 RT-LAMP reactions per condition without reverse transcriptase, targeting an alternative template site within the SARS-CoV-2-N-gene RNA (approximately 30 template molecules per reaction). Reactions were performed without or with LNA modifications in the F3 and/or B3 LAMP primers, as indicated on the left side of the panel. ‘+’ signs indicate LNA modifications in the subsequent base. All primer sequences are indicated below. Numbers of reactions interpreted as positive are indicated on the right side of the gel.

The examples illustrate the invention.

### Example 1—Material and Methods

1) LNA-LAMP reaction (used in FIGS. 1, 2, 4, 6 and 9):

Except where indicated otherwise, 12.5 μl LNA-LAMP reactions contained the following components:
1.25 μl Isothermal Reaction Buffer (New England Biolabs, B0537S)
0.44 μl dNTP mix 10 mM (NEB, N0447L)
2.8 μl 1 M Tris-HCl pH 8.5 (Jena Biosciences)
0.75 μl MgSO₄ 100 mM (NEB, B1003S)
0.0125 μl pUC19 plasmid DNA 1 μg/μL (NEB, N3041L)
0.4 μM FIP primer(s) (see figures)
0.4 μM BIP primer(s) (see figures)
0.2 μM F3 primer(s) (see figures)
0.2 μM B3 primer(s) (see figures)
0.4 μMLF primer(s) (see figures)
0.4 μM LB primer(s) (see figures) 4.15 μl template RNA in heat-inactivated lysis buffer, containing:
   300 mM Tris-HCl pH 8.5
   2.5 M Trimethylglycine (Sigma, 61962-50G)
   2.7 units/ml Proteinase K (NEB, P8107S)
0.5 μl wild-type Bst-LF-Idaho polymerase (1 mg/mL)
fill-up to 12.5 μl, water

LAMP reactions were incubated at 65° C. for 80 minutes.

2) PCR amplification and detection of LAMP products (used in FIGS. 1, 2, 4, 6 and 9):

LAMP reactions were diluted 20-fold in water.

10 μl PCR reactions contained the following components:
5 μL NEBNext 2× Master Mix (New England Biolabs)
0.5 μM fwd primer (see figures)
0.5 μM rev primer (see figures)
1 μl diluted LAMP reaction

PCR reactions were temperature-cycled using the following protocol:

| | | | |
|---|---|---|---|
| 3 | minutes | 98° | C. |
| 20 | seconds | 98° | C. (20 repeats) |
| 20 | seconds | 65° | C. (20 repeats) |
| 30 | seconds | 72° | C. (20 repeats) |
| 3 | minutes | 72° | C. |

| | | |
|---|---|---|
| Hold | 4° | C. |

2 μl of each PCR reaction were run on a 2% agarose gel for 30 minutes at 100 V, visualized using SYBR Safe, and imaged using a BioRad digital gel imaging system.

3) Clinical RT-qPCR (used in FIG. 3, 5):

Swabs were rehydrated for at least 10 seconds in 600 μl−1 ml LNA-LAMP lysis buffer (300 mM Tris-HCl pH 8.5, 2.5 M Trimethylglycine, 2.7 units/ml Proteinase K) or other lysis buffer (see figure), and heated for up to 15 minutes to 95° C. Viral RNA was extracted using the Chemagic Prime Viral DNA/RNA 300 kit (PerkinElmer) on a Chemagic Prime 8 system (PerkinElmer). The viral sample (150-290 μl) was mixed with 10 μl of the internal control sample and 300 μl lysis buffer. Extraction was performed according to the manufacturer's protocol, and viral RNA was eluted in 45 μl elution buffer for subsequent analysis. Detection of viral RNA using one-step real-time reverse-transcription PCR was performed according to Corman et al. (2020)18 with the iTaq Universal Probes One-Step kit (BioRad), using 5 μl of eluate per reaction and primers and probes against the E gene (E_Sarbeco_F1: ACAGGTACGTTAATAGTTAATAGCGT (SEQ ID NO: 2), E_Sarbeco_R2: ATATTGCAGCAGTACGCACACA (SEQ ID NO: 3), E_Sarbeco_P1: FAM-ACACTAGCCATCCTTACTGCGCTTCG-BBQ (SEQ ID NO: 4); TIB MolBiol). Spike-in RNA of the bacteriophage MS2 served as an internal control and was detected using the Luna Universal Probe One-Step RT-qPCR kit (New England Biolabs) using 2 μl of eluate and corresponding primers and probes (MS2_F: TGCTCGCGGATACCCG (SEQ ID NO: 5), MS2_R: AACTTGCGTTCTCGAGCGAT (SEQ ID NO: 6), MS2_P: YAK-ACCTCGGGTTTCCGTCTTGCTCGT-BBQ (SEQ ID NO: 7); TIB MolBiol). The reactions for the E gene and internal control were performed using dual detection of FAM and YAK/VIC in a Lightcycler 480 (Roche).

4) Comparison of LNA-LAMP to clinical RT-qPCR (FIG. 5):

For each clinical lysate, RNA extraction/RT-qPCR and four LNA-LAMP reactions were performed in parallel, with a total volume of 25 μl per LNA-LAMP reaction containing 8.3 μl lysate each. LNA-LAMP results were counted as positive if two or more out off our LNA-LAMP reactions yielded detectable LAMP products as analysed by sequencing (LAMP-Seq).

5) 2-step RT-PCR (used in FIG. 7)

10 μl RT reactions contained the following components:
1 μl Isothermal Reaction Buffer (New England Biolabs, B0537S)
0.35 μl dNTP mix 10 mM (NEB, N0447L)
2.24 μl 1 M Tris-HCl pH 8.5 (Jena Biosciences)
0.6 μl MgSO₄ 100 mM (NEB, B1003S)
0.4 μM RT primer (G+TA+AG+AA+CA+CCTGTGCCTGTTA (SEQ ID NO: 8), +indicates subsequent LNA base, IDT)
4 μl lysis buffer (300 mM Tris-HCl pH 8.5, 2.5 M Trimethylglycine)
0.4 μl wild-type Bst-LF-Idaho polymerase (1 mg/mL)
1 μl SARS-CoV-2 genomic RNA purified from a positive human oropharyngeal swab sample

RT reactions were incubated at 65° C. for 80 minutes.

10 μl PCR reactions contained the following components:
5 μl NEBNext 2×PCR Master Mix (New England Biolabs)
0.5 μM fwd primer

(ACACTCTTTCCCTACACGACGCTCTTCCGATCTAGACTTTTTAGGTCCA
CAAACA (SEQ ID NO: 9), IDT)

0.5 μM rev primer

(TGACTGGAGTTCAGACGTGTGCTCTTCCGATCTCCGGTAGCACACCTTG
TAATGGT (SEQ ID NO: 10), IDT)

1 μl RT reaction
Fill up to 10 μl, water

PCR reactions were temperature-cycled using the following protocol:

| | | | |
|---|---|---|---|
| 3 | minutes | 98° | C. |
| 20 | seconds | 98° | C. (35 repeats) |
| 20 | seconds | 65° | C. (35 repeats) |
| 30 | seconds | 72° | C. (35 repeats) |
| 3 | minutes | 72° | C. |

| | | |
|---|---|---|
| Hold | 4° | C. |

6) 1-step RT-PCR (used in FIG. 7)

10 μl RT reactions contained the following components:
1 μl Isothermal Reaction Buffer (New England Biolabs, B0537S)
0.35 μl dNTP mix 10 mM (NEB, N0447L)
2.24 μl 1 M Tris-HCl pH 8.5 (Jena Biosciences)
1.20 μl MgSO₄ 100 mM (NEB, B1003S)
0.4 μM RT primer (G+TA+AG+AA+CA+CCTGTGCCTGTTA (SEQ ID NO: 8), +indicates subsequent LNA base, IDT)
3.4 μl lysis buffer (300 mM Tris-HCl pH 8.5, 2.5 M Trimethylglycine)
0.4 μl wild-type Bst-LF-Idaho polymerase (1 mg/mL)
1 μl SARS-CoV-2 genomic RNA purified from a positive human oropharyngeal swab sample

RT reactions were kept on ice and the following 10 μl PCR reaction was added using the KOD Hot Start DNA Polymerase kit (Novagen, 71086):
1 μl 10× Buffer for KOD Hot Start DNA Polymerase
0.6 μL MgSO₄ (25 mM)
1 μl dNTP mix (2 mM each)
0.3 μM fwd primer

(ACACTCTTTCCCTACACGACGCTCTTCCGATCTAGACTTTTTAGGTCCA
CAAACA (SEQ ID NO: 9), IDT)

0.3 μM rev primer

(TGACTGGAGTTCAGACGTGTGCTCTTCCGATCTCCGGTAGCACACCTTG
TAATGGT (SEQ ID NO: 10), IDT)

0.2 μl KOD Hot Start DNA Polymerase (1 U/μl)
Fill up to 10 μl, water

RT-PCR reactions were temperature-cycled using the following protocol:

| | | | |
|---|---|---|---|
| 30 | minutes | 65° | C. |
| 2 | minutes | 95° | C. |
| 20 | seconds | 95° | C. (35 repeats) |
| 10 | seconds | 65° | C. (35 repeats) |
| 10 | seconds | 70° | C. (35 repeats) |
| 3 | minutes | 70° | C. |

| | | |
|---|---|---|
| Hold | 4° | C. |

7) Application of piercing funnel device for scalable swab sample acquisition (related to FIG. 8):

96-well round-well deep-well plates (Eppendorf) were filled with 700 μl lysis buffer per well, containing 300 mM Tris-HCl pH 8.5, 2.5 M Trimethylglycine, and 2.7 units/ml Proteinase K. Positive control RNA was added to one well per plate (H12, 169,000 molecules per well). The plate was heat-sealed with a pierceable aluminium foil and transported to the testing site. After self-registration, participants presented at the center. Supported by visual and audio cues by a custom-designed software, trained staff pierced one position on the plate with the disposable piercing funnel device, obtained an oropharyngeal swab, and introduced it through the funnel into the well. After submerging it for at least 10 seconds assisted by an automated timer, the swab was retracted into the funnel, and both were removed together. The well was subsequently sealed with a silicone plug. After transport to the pre-LAMP-lab, lysate plates were sterilized in a thermo block at 95° C. Up to 8.3 μl of lysate were transferred to a ready-made barcoded LNA-LAMP 384-well plate. Plates were heated in a water bath to 65° C. for 80 minutes in a separate post-LAMP-lab, and reactions were pooled using multichannel pipetting or centrifugation. Positive LAMP reactions were identified by sequencing (https://www.biorxiv.org/content/10.1101/2020.04.06.025635v2).

8) Protein sequence of Bst-LF-Idaho polymerase (used throughout experiments)

(SEQ ID NO: 30)
MKHHHHHHSAGLEVLFQGP MESPSSEEEKPLAKMAFTLAD
RVTEEMLADKAALVVEVVEE NYHDAPIVGIAVVNEHGRFF
LRPETALADPQFVAWLGDET KKKSMFDSKRAAVALKWKGI
ELCGVSFDLLLAAYLLDPAQ GVDDVAAAAKMKQYEAVRPD
EAVYGKGAKRAVPDEPVLAE HLVRKAAAIWELERPFLDEL
RRNEQDRLLVELEQPLSSIL AEMEFAGVKVDTKRLEQMGK
ELAEQLGTVEQRIYELAGQE FNINSPKQLGVILFEKLQLP
VLKKTKTGYSTSADVLEKLA PYHEIVENILHYRQLGKLQS
TYIEGLLKVVRPDTKKVHTI FNQALTQTGRLSSTEPNLQN
IPIRLEEGRKIRQAFVPSES DWLIFAADYSQIELRVLAHI
AEDDNLMEAFRRDLDIHTKT AMDIFQVSEDEVTPNMRRQA
KAVNFGIVYGISDYGLAQNL NISRKEAAEFIERYFESFPG
VKRYMENIVQEAKQKGYVTT LLHRRRYLPDITSRNFNVRS
FAERMAMNTPIQGSAADIIK KAMIDLNARLKEERLQAHLL
LQVHDELILEAPKEEMERLC RLVPEVMEQAVTLRVPLKVD
YHYGSTWYDAK*

9) Bst-LF-Idaho polymerase protein expression and purification (used throughout experiments)

Bst polymerase large-fragment from a Geobacillus strain sampled in Idaho (Kiefer, J. R. et al. Crystal structure of a thermostable Bacillus DNA polymerase I large fragment at 2.1 A resolution. Structure 5, 95-108 1997) was cloned into a pET vector with a N-terminal His6-3C-tag. Recombinant protein was expressed in E. coli BL21 Rosetta (DE3) cells in TB auto induction media supplemented with 17 mM KH₂PO₄, 72 mM K₂HPO₄, 1.5% lactose, 0.05% glucose, and 2 mM MgSO₄ at 18° C. overnight. Cells were harvested by centrifugation and resuspended in lysis buffer (50 mM Tris/HCl pH 8.0, 1 M NaCl, 20 mM imidazol, 10% glycerol) followed by sonication. The lysate was cleared in a Beckman-Coulter Avanti JNX-26 centrifuge with a JA-25.50 rotor (20.000 rpm for 30 min at 4° C.) and applied to a HisTrap FF column (GE Healthcare). After washing with 10 column volumes of lysis buffer, protein was eluted in elution buffer (50 mM Tris/HCl pH 8.0, 0.5 M NaCl, 200 mM imidazol, 10% glycerol). Fractions of the main peak were pooled and diluted 1:10 with IEX loading buffer (20 mM Tris/HCl, 100 mM NaCl, 10% glycerol) and the affinity tag was removed using 1:100 3C protease overnight at 4° C. Protein was loaded onto a reverse HisTrap FF column coupled to a HiTrapQ HP column (GE Healthcare). After loading, the HisTrap column was removed and protein was eluted from the HiTrapQ column with 25% IEX elution buffer (20 mM Tris/HCl, 1 M NaCl, 10% glycerol). Fractions of the main peak were pooled and diluted 1:5 with Heparin loading buffer (20 mM Tris/HCl, 100 mM NaCl, 10% glycerol). Sample was loaded onto a HiPrep Heparin FF column (GE Healthcare) and eluted using 40% Heparin elution buffer (20 mM Tris/HCl, 1 M NaCl, 10% glycerol). Fractions of the main peak were concentrated using Amicon filters (Millipore) and applied to size exclusion chromatography using a Superdex 200 PrepGrade column (GE Healthcare) equilibrated with SEC buffer (25 mM Tris/HCl pH 8.0, 250 mM KCl). Fractions of the main peak were pooled, concentrated to 1 mg/ml using Amicon filters and stored in 1 ml aliquots in storage buffer (10 mM Tris/HCl pH 7.5, 100 mM KCl, 1 mM DTT, 0.1 mM EDTA, 50% glycerol) at −20° C.

### Example 2—Efficient LNA-LAMP without a Reverse Transcriptase

LNA-modified primers can improve hybridization to RNA targets and result in more efficient reverse transcription. To determine if LNA modifications in the F3 and B3 LAMP primers would allow to perform sensitive RT-LAMP reactions in the absence of a reverse transcriptase, reaction conditions with or without LNA modifications, with or without reverse transcriptase, and with or without template were conducted (FIG. 1). Either reverse transcriptase, or LNA-modified F3 and B3 primers were required for any amplification products to be detected. Of note, both working configurations resulted in a similar sensitivity. An all-negative control reaction including an RNase-A digestion of template RNA before LAMP confirmed that RNA was detected rather than a putative contaminating DNA species. Furthermore, the use of LNA-modifications did not lead to unspecific reaction products as shown by negative control conditions lacking template RNA. This result demonstrates the possibility to perform LAMP reactions utilizing only the reverse transcriptase activity of Bst-LF-Idaho polymerase by using LNA modified F3 and B3 primers, thus minimizing reaction complexity and cost per sample.

### Example 3—Optimization of LNA Modification Patterns in RT-LAMP

To optimize the positioning and extent of LNA primer modifications used for LNA-LAMP, experiments comparing different modification patterns were performed (FIG. 2). 3-6 LNA modifications per F3 and B3 primer yielded the highest sensitivity. An alternating pattern of LNA-modified and unmodified bases was found to be required for high sensitivity. The region bearing LNA-modified positions can equally be placed at the very 5′ end of primers, or starting at the second position from the 5′ end, or in the middle of the primers, but preferably not at the 3′ end of the primers. The highest sensitivity was detected using five LNA modifications alternating with unmodified bases, starting at the second positions from the 5′ end of the F3 and B3 primers, respectively. LNA modification of the FIP and BIP, or LB and LF primers interfere with sensitive RNA detection of LAMP reactions with reverse transcriptase present, potentially interfering with the re-amplification of DNA products after incorporating LNA modifications.

### Example 4—Lysis Buffers and Conditions Suitable for Extraction-Free LNA-LAMP Testing

For scalable application of LNA-LAMP, a resuspension buffer and protocol are needed to circumvent a laborious RNA extraction step. To define such a protocol, pairs of oropharyngeal swab samples from SARS-CoV-2 positive patients were used to compare buffers and conditions to standard resuspension of swabs in DPBS and no heating (FIG. 3). Viral RNA was extracted and quantified using E-gene specific RT-qPCR, with resulting Ct-values indicated. Heating samples in a buffer containing 300 mM Tris pH 8.5, 2.7 u/ml Proteinase K, and 2.5 M Trimethylglycine (Betaine) but no detergents like Tween-20 or Triton-X100 yielded a similar amount of viral RNA as resuspension in DPBS without heating, and was therefore used for further experiments.

### Example 5—Resin-Assisted Sensitive Detection of Viral RNA by LNA-LAMP from Unpurified Human Samples

For scalable application of LNA-LAMP, a laborious RNA extraction step should be circumvented. Therefore, a suitable condition for resuspending swab samples, lysing viral particles, and inactivating RNases and Proteinase K is required which allows direct introduction of lysates into LNA-LAMP. As shown in FIG. 4, pre-incubation of crude lysates with Weak Acid Ion Exchange Beads and Activated Granular Carbon increased the sensitivity of LNA-LAMP when detecting synthetic SARS-CoV-2 RNA. Adding granular resin during lysis does not increase liquid handling complexity, as granules sink to the bottom of the vessel and are not transferred to the LNA-LAMP reaction by pipetting.

### Example 6—Performance of Purification-Free LNA-LAMP Against a Standard Clinical Extraction-RT-qPCR Pipeline

Validating LNA-LAMP on 676 residual swab samples from clinical testing using automated liquid handling robots, LNA-LAMP displayed a sensitivity of 100% among 58 positive samples with Ct values below 33 in parallel RT-qPCR testing (FIG. 5) and a specificity of 99.7%. Of 16 samples with Ct-values between 33 and 36, 15 samples were identified as positive by LNA-LAMP (sensitivity: 93.8%), while 24 weakly-positive samples (Ct above 36) were detected stochastically.

### Example 7—Evaluation of LNA-LAMP-Mediated Detection of Influenza-A Genomic RNA, Optionally Performed Simultaneously with SARS-CoV-2 Detection in a Single Reaction

Multiplexing of several target sequences in a single LNA-LAMP reaction might enable differential diagnostics of multiple pathogens as well as detection of specific viral variants at scale. For establishing simultaneous detection of multiple pathogens in a single LNA-LAMP reaction, primers targeting the Influenza-A viral genome were tested on synthetic template RNA by LNA-LAMP, revealing a similar performance as observed for SARS-CoV-2 diagnostics using LNA-LAMP (FIG. 6). Multiplexing LNA-LAMP-mediated amplification of Influenza-A RNA and SARS-CoV-2 RNA sequences in a single LNA-LAMP reaction did not affect the sensitivity for Influenza-A RNA, thus enabling parallel detection of two or more pathogens by LNA-LAMP.

### Example 8—Targeted RNA Sequencing of the SARS-CoV-2 S-Gene Using LNA-Assisted Reverse Transcription with a Bst Polymerase Using a 1-Step or 2-Step RT-PCR Protocol

For discrimination of pathogen variants of concern, a scalable targeted sequencing protocol is instrumental. LNA modifications enable Bst-LF-Idaho polymerase to efficiently perform reverse transcription (RT) in LNA-LAMP as detailed above. Therefore, we investigated if it could also enable RT in a 2-step or 1-step RT-PCR procedure, with the goal of amplifying a specific stretch of viral genomic RNA to a sufficient amount and purity for Next-Generation-Sequencing. An RT reaction with Bst-LF-Idaho polymerase and with an LNA-modified RT primer and subsequent PCR with a pair of unmodified target-specific primers successfully amplified product DNA of the expected size in a template RNA-dependent fashion (FIG. 7). Furthermore, combining the LNA-modified RT primer, Bst-LF-Idaho polymerase, PCR primers, and further PCR reaction components into a 1-step reaction gave rise to a correct product as well, potentially simplifying library preparation to a single reaction setup. Sequencing the product of the 2-step LNA-enabled RT-PCR protocol on an Illumina Misesq™ sequencer yielded in >20% of raw sequencing reads matching the target (SARS-CoV-2 S-gene), and the sequencing result matched variant of concern B.1.1.7 (UK variant).

### Example 9—Piercing Funnel Device for Fast and Contamination-Free Sampling in a Robotics-Compatible Format

For scalable, reliable, and safe processing of swab samples, an automation-compatible sample format should be employed from the earliest possible step of the testing procedure. To enable swab elution, lysis, and storage in a robot-compatible standard 96-well plate format, plates can be pre-filled with lysis buffer and sealed with a pierceable aluminium foil. With the piercing funnel device depicted in FIG. 8, an individual well of the plate is pierced. The device is left in the pierced cavity to enable safe insertion of an inoculated swab through the piercing funnel device into the well, where agitating in the lysis buffer can be performed for a defined time. Retracting the wet portion of the swab into the piercing funnel device allows to safely remove swab and funnel together. Lastly, the well is closed, e.g., using a silicone plug or plastic cap. The piercing funnel design depicted was 3D-printed in plastics and optimized to ensure a fast, safe, and convenient way to extract sample material from human swabs into lysis buffer, while minimizing cross-contamination of neighbouring wells or other parts of the working environment.

### Example 10—Determination of Required LNA-Modified Primers in RT-LAMP without Using a Reverse Transcriptase

To determine whether LNA-modifications are required for both F3 and B3 primers, or just for one of these primers, experiments were performed using different combinations of LNA-modified primers. These experiments revealed that the combination of an LNA-modified B3-Primer with an unmodified F3-primer was able to yield a similar sensitivity of detecting two independent RNA template regions of the SARS-CoV-2 N-gene, as compared to using both F3 and B3 primers with LNA modifications (FIGS. 9a and b). On the other hand, RT-LAMP reactions with LNA-modified F3-primer and unmodified B3-primer did not provide high sensitivity RNA detection. The experiments comprised two independent target regions of the SARS-CoV-2 N-gene. The second target region (FIG. 9b) was positioned such as to enable differentiation between several SARS-CoV-2 variants from the sequencing data.

### REFERENCES

1. E. Dong, H. Du, L. Gardner, An interactive web-based dashboard to track COVID-19 in real time. The Lancet. Infectious diseases 20, 533-534 (2020); published online EpubMay (10.1016/S1473-3099(20)30120-1).
2. M. Chinazzi, J. T. Davis, M. Ajelli, C. Gioannini, M. Litvinova, S. Merler, Y. P. A. Pastore, K. Mu, L. Rossi, K. Sun, C. Viboud, X. Xiong, H. Yu, M. E. Halloran, I. M. Longini, Jr., A. Vespignani, The effect of travel restrictions on the spread of the 2019 novel coronavirus (COVID-19) outbreak. Science 368, 395-400 (2020); published online EpubApr 24 10 (10.1126/science.aba9757).
3. R. Li, S. Pei, B. Chen, Y. Song, T. Zhang, W. Yang, J. Shaman, Substantial undocumented infection facilitates the rapid dissemination of novel coronavirus (SARS-CoV-2). Science 368, 489-493 (2020); published online EpubMay 1 (10.1126/science.abb3221).
4. Y. Bai, L. Yao, T. Wei, F. Tian, D. Y. Jin, L. Chen, M. Wang, Presumed Asymptomatic 15 Carrier Transmission of COVID-19. Jama, (2020); published online EpubFeb 21 (10.1001/jama.2020.2565).
5. J. Taipale, P. Romer, S. Linnarsson, Population-scale testing can suppress the spread of COVID-19. medRxiv: the preprint server for health sciences, 2020.2004.2027.20078329 20 (2020)10.1101/2020.04.27.20078329).
6. J. Peto, Covid-19 mass testing facilities could end the epidemic rapidly. BMJ 368, m1163 (2020)10.1136/bmj.m1163).
7. J. S. Bloom, E. M. Jones, M. Gasperini, N. B. Lubock, L. Sathe, C. Munugala, A. S. Booeshaghi, O. F. Brandenberg, L. Guo, S. W. Simpkins, I. Lin, N. LaPierre, D. Hong, Y. Zhang, G. Oland, B. J. Choe, S. Chandrasekaran, E. E. Hilt, M. J. Butte, R. Damoiseaux, A. R. Cooper, Y. Yin, L. Pachter, O. B. Garner, J. Flint, E. Eskin, C. Luo, S. Kosuri, L. Kruglyak, V. A. Arboleda, Swab-Seq: A high-throughput platform for massively scaled up SARS-CoV-2 testing. medRxiv: the preprint server for health sciences, 2020.2008.2004.20167874 (2020)10.1101/2020.08.04.20167874).
8. V. L. Dao Thi, K. Herbst, K. Boerner, M. Meurer, L. P. Kremer, D. Kirrmaier, A. Freistaedter, D. Papagiannidis, C. Galmozzi, M. L. Stanifer, S. Boulant, S. Klein, P. Chlanda, D. Khalid, I. Barreto Miranda, P. Schnitzler, H. G. Krausslich, M. Knop, S. 10 Anders, A colorimetric RT-LAMP assay and LAMP-sequencing for detecting SARS-CoV-2 RNA in clinical samples. Science translational medicine 12, (2020); published online EpubAug 12 (10.1126/scitranslmed.abc7075).
9. P. James, D. Stoddart, E. D. Harrington, J. Beaulaurier, L. Ly, S. Reid, D. J. Turner, S. Juul, LamPORE: rapid, accurate and highly scalable molecular screening for SARS-CoV-2 15 infection, based on nanopore sequencing. medRxiv: the preprint server for health sciences, 2020.2008.2007.20161737 (2020)10.1101/2020.08.07.20161737).

1. A method for the detection of a target nucleic acid sequence in a sample, wherein the method comprises:
a) optionally contacting the sample with a lysis buffer under conditions wherein the sample is lysed,
b) optionally heating the lysis buffer;
c) subjecting the lysate as obtained after step a) and optionally after step b) or the sample to an isothermal amplification reaction at a temperature of 30 to 75° C., preferably 45 to 75°, more preferably 60 to 70° C., even more preferred about 65° C. with at least two primers specifically amplifying the target nucleic acid sequence, wherein the nucleotides of at least one primer comprise at least two locked nucleic acids (LNAs) which are not directly adjacent to each other within the nucleotides of the at least one primer, and
wherein the isothermal amplification reaction comprises a DNA polymerase with reverse transcriptase activity and strand displacement activity and does not comprise a thermostable reverse transcriptase; and
d) detecting the presence of the target nucleic acid sequence in the amplification product obtained after or during step d).

2. The method of claim 1, wherein in step c) the isothermal amplification reaction with at least two primers specifically amplifying the target nucleic acid sequence is a reverse transcriptase loop-mediated isothermal amplification (RT-LAMP) reaction with at least four primers specifically amplifying the target nucleic acid sequence, wherein two primers are outer primers and at least two primers are inner primers, and wherein the at least one primer comprising the at least two locked nucleic acids (LNAs) is one, preferably both of the outer primers.

3. The method of claim 2, wherein the RT-LAMP reaction is a LAMP-Seq reaction.

4. The method of claim 1, wherein the DNA polymerase with reverse transcriptase activity and strand displacement activity is selected from
(i) a Bacillus stearothermophilus (Bst) DNA Polymerase I, a large fragment of Bst DNA Polymerase I, or a homolog thereof retaining 5′→3′ DNA polymerase activity, reverse transcriptase activity and strong strand displacement activity of the large fragment of Bst DNA Polymerase I, and/or
(ii) the exo-variant of the E. coli DNA Polymerase I and Bsu DNA Polymerase, Large Fragment.

5. The method of claim 1 wherein the lysis buffer comprises weak-acid-ion-exchange particles and/or granular activated carbon (GAC).

6. The method of claim 5, wherein the GAC are coconut shell-based GAC and preferably washed coconut shell-based GAC.

7. The method of claim 5, wherein per 100 μl of lysis buffer 10-60 milligram, preferably 20-50 milligram and most preferably about 35 milligram of weak-acid-ion-exchange particles and/or GAC are comprised in the lysis buffer.

8. The method of claim 1, wherein the lysis buffer is an aqueous lysis buffer comprising 100 to 1000 mM Tris pH 8.0 to pH 9.0, preferably about pH 8.5, 1 to 6 M Trimethylglycin, and 1 to 10 u/ml Proteinase.

9. The method of claim 1, wherein the sample is a swab sample, preferably an oropharyngeal, nasopharyngeal, anal, buccal, skin or mid-nasal swab sample.

10. The method of claim 1, wherein the sample is or comprises a body fluid, wherein the body fluid is preferably saliva, sputum, feces, urine, blood, serum, plasma, semen, vaginal fluid, mucus, tears, and milk.

11. The method of claim 1, wherein in step b) the lysis buffer is heated to 60 to 100° C., preferably to about 95° C.

12. The method of claim 1, wherein the sample is transferred from the lysis buffer to the amplification reaction by the same tool that was used to collect the sample.

13. The method according to claim 12, wherein the tool is a swab, brush, toothpick, plastic stick, or spoon.

14. The method according to claim 1 wherein the target nucleic acid sequence is a pathogenic nucleic acid sequence, preferably a viral nucleic acid sequence.

15. The method according to claim 14, wherein the viral nucleic acid sequence is a betacoronavirus nucleic acid sequence, influenza nucleic acid sequence or a RSV nucleic acid sequence, wherein the betacoronavirus is preferably selected from SARS-CoV-2, MERS-CoV, SARS-CoV-1, OC43, and HKU1, and is most preferably SARS-CoV-2.
